(19) United States
(12) Patent Application Publication
(10) Pub. No.: US 2009/0270815 A1
(43) Pub. Date: Oct. 29, 2009

(54) Catheter Priming System

(75) Inventors: Andrea Stamp, Gloucester, MA (US); Richard Gambale, Tyngsboro, MA (US); Jay Caplan, Belmont, MA (US); Robert McNeil, San Mateo, CA (US); Jerry Brightbill, Newton, MA (US)

Correspondence Address: FISH & RICHARDSON PC, P.O. BOX 1022, MINNEAPOLIS, MN, 55440-1022, US

(73) Assignee: InfraReDx, Inc., Burlington, MA (US)

(21) Appl. No.: 12/111,457

(22) Filed: Apr. 29, 2008

(51) Int. Cl. A61M 25/14

(52) U.S. Cl. 604/249; 604/256

(57) ABSTRACT

A catheter includes a longitudinally extending catheter body having proximal and distal ends. The catheter body defines a lumen extending longitudinally from a first port defined at the proximal end, through the body, to a second port defined at the distal end. The catheter includes a valve for controlling fluid flow through the second port.

### TECHNICAL FIELD

This disclosure relates to catheters.

### BACKGROUND

A catheter is a tube that can be inserted into a body cavity, duct or vessel to allow drainage or injection of fluids or access by surgical instruments. Catheterization may be used to drain urine from a urinary bladder, to drain accumulated fluid (e.g. an abdominal abscess), administer intravenous fluids, medication, directly measure blood pressure or intracranial pressure, to perform angioplasty, angiography, balloon septostomy, and balloon sinuplasty, inter alia, for example. A balloon catheter is a type of catheter with an inflatable “balloon” at its tip. The balloon is used during a catheterization procedure to enlarge a narrow opening or passage within the body.

### SUMMARY

In one aspect, a catheter includes a longitudinally extending catheter body having proximal and distal ends. The catheter body defines a lumen extending longitudinally from a first port defined at the proximal end, through the body, to a second port defined at the distal end. The catheter includes a valve for controlling fluid flow through the second port.

Implementations of this aspect of the disclosure may include one or more of the following features. In some implementations, the valve includes a valve element pivotally attached to the catheter body and extending into the lumen. The valve also includes a valve seat defined by the catheter body in the lumen and configured to receive the valve element. The valve element is configured to transition between a first state in sealing engagement with the valve seat, and a second state spaced from the valve seat for permitting fluid flow through the second port in response to pressure in the lumen. The valve element is biased to maintain the first state.

In some implementations, the valve includes a compliant flap secured to the catheter body in the lumen and a valve seat defined by the catheter body in the lumen. The valve seat is configured to receive the flap. The flap is configured to maintain a first state in sealing engagement with the valve seat and to elastically deflect into a second state spaced from the valve seat for permitting fluid flow through the second port when pressure in the lumen exceeds a threshold pressure.

In some examples, the valve includes at least first and second compliant flaps secured opposite each other in the lumen. The flaps are elastically deformable to move between a first state in sealing engagement with each other to inhibit fluid flow through the second port, and a second state that allows fluid flow through the second port out of the lumen when pressure in the lumen exceeds a threshold pressure. The first and second compliant flaps each have proximal and distal ends with a broad side extending therebetween. The proximal ends of the flaps are secured circumferentially to the inner wall of the lumen. The flaps extend toward each other and meet along their broad sides when in the first position.

The valve, in some examples, includes an elastic tube surrounding the catheter body in a first state in sealing engagement with an outer surface of the catheter body to temporarily seal the second port. The tube is configured to distend into a second state that allows fluid flow out of the second port when pressure in the lumen exceeds a threshold pressure. The elastic tube has proximal and distal ends, with the distal end being secured to the catheter body.

In some implementations, the valve includes a valve element disposed in the lumen for movement, a valve seat defined by the catheter body in the lumen, and a spring for biasing the valve element toward a first position in sealing engagement with the valve seat. The valve element is operable to move from the first position in sealing engagement with the valve seat to a second position spaced from the valve seat for permitting fluid flow through the second port. The valve element moves from the first position to the second position when pressure in the lumen exceeds a threshold pressure.

The valve, in some examples, includes first and second compliant bladders mounted opposite each other in the lumen and disposed in a first position in sealing engagement with each other to inhibit fluid flow through the second port. The first and second compliant bladders move to a second position for permitting fluid flow between the bladders and through the second port when pressure in the lumen exceeds a threshold pressure.

In some implementations, the valve includes a membrane disposed across the lumen. The membrane defines an aperture configured to remain closed in a first position to inhibit fluid flow through the second port. The aperture opens to a second position for permitting fluid flow through the second port when pressure in the lumen exceeds a threshold pressure.

In some examples, the catheter body defines at least one axial groove in a wall of the lumen. The groove extends from the proximal end of the catheter body to the distal end of the catheter body. The catheter body may also, or instead, define at least one axial protrusion in a wall of the lumen. The protrusion extends from the proximal end of the catheter body to the distal end of the catheter body.

In another aspect, a catheter includes a longitudinally extending catheter body having proximal and distal ends. The catheter body defines a first lumen extending longitudinally between a first port, defined at the proximal end, and a second port, defined at the distal end. The catheter body also defines a second lumen extending from a side of the catheter body to the distal end of the catheter body. The second port provides fluid communication between the first and second lumens. An elastomeric tube is disposed in the second lumen in a first state in which it seals a wall of the second lumen to inhibit fluid flow through the second port. The tube elastically deflects to a second state spaced from the wall of the second lumen for permitting fluid flow through the second port when pressure in the first lumen exceeds a threshold pressure.

In yet another aspect, a catheter includes a longitudinally extending catheter body having proximal and distal ends. The catheter body defines a lumen extending longitudinally from a first port, defined at the proximal end, through the body to a second port, defined at the distal end. A filter is disposed in the lumen for controlling fluid flow through the second port. The filter is configured to prevent blood flow therethrough. The filter may define fluid passageways therethrough having a diameter less than about 8 microns.

In another aspect, a catheter includes a longitudinally extending catheter body having proximal and distal ends. The catheter body defines a lumen extending longitudinally from a first port, defined at the proximal end, through the body to a second port, defined at the distal end, and a valve seat. A valve in fluid communication with the second port controls fluid flow through the second port. The valve includes a valve element disposed within the lumen for transition between a first state in sealing engagement with the valve seat to inhibit fluid flow through the second port, and a second state spaced from the valve seat for permitting fluid flow through the second port. The valve includes a valve element operator that, when engaged by a deliverable instrument received by the lumen, causes a transition of the valve element from the first position to the second position.

Implementations of this aspect of the disclosure may include one or more of the following features. In some implementations, the valve element is secured in the lumen to the catheter body and extends circumferentially along a wall of the lumen over the second port to a distal tip. The valve element is torsionally biased against the lumen wall in sealing engagement with the valve seat defined by the lumen wall. Circumvolution of the valve element operator disposed at the distal tip of the valve element causes the valve element to elastically deform from the first position to the second position, permitting fluid flow through the second port. A distal tip of the received deliverable instrument is configured to engage the valve element operator, such that torsional movement of the received deliverable instrument is translated to the valve element operator.

In some examples, the valve seat is an orifice defined by the catheter body in the lumen. The valve element is disposed distally of the orifice and spring biased toward sealing engagement with the valve seat to occlude the orifice. The valve element operator extends through the orifice for actuation of the valve by the received deliverable instrument.

In some implementations, the valve element is a lumen plug slidably disposed within the lumen for movement between a first position that permits fluid flow through the second port, and a second position in sealing engagement with the valve seat defined by a wall of the lumen, occluding the lumen. The lumen plug may define a fluid channel extending from a longitudinal proximal end of the lumen plug to a lateral side of the lumen plug. The fluid channel is in fluid communication with the second port when the lumen plug is in the first position. The lumen plug may be biased by a spring toward sealing engagement with the valve seat.

The valve element may be a gate pivotally attached to the catheter body in the lumen. The gate swings from the first position, in sealing engagement with the valve seat, to the second position when moved by the received deliverable instrument. A side wall of the catheter body defines the second port. The valve element extends along a wall of the lumen for sealing engagement with the valve seat defined by the lumen wall. The valve element operator extends at least partially across the lumen for actuation by the received deliverable instrument.

In some implementations, the valve element is disposed in the lumen and elastically deformed by the deliverable instrument when moved from the first position, in sealing engagement with the valve seat, to the second position, permitting fluid flow through the second port. The valve element may be an ovular tube having inner and outer walls. A portion of the inner wall of the tube defines the valve element operator. The received deliverable instrument engages the valve element operator to elastically alter a geometry of the tube to the second position for permitting fluid flow though the second port.

In some examples, the valve element is an inflatable bladder disposed in the lumen. The bladder is operable to distend between a first position in sealing engagement with the valve seat and the second position for allowing fluid flow through the second port. The catheter body defines a bladder inflation channel in fluid communication with the bladder to control bladder inflation.

The catheter body may define at least one axial groove in a wall of the lumen. The groove extends from the proximal end of the catheter body to the distal end of the catheter body. The catheter body may also, or instead, define at least one axial protrusion in a wall of the lumen. The protrusion extends from the proximal end of the catheter body to the distal end of the catheter body.

In another aspect, a catheter includes a longitudinally extending catheter body having proximal and distal ends. The catheter body defines a lumen extending longitudinally from a first port, defined at the proximal end, through the body to a second port, defined at the distal end. The catheter includes a valve for controlling fluid flow through the second port. The valve includes an elastic tube surrounding the catheter body in a first state for sealing an outer surface of the catheter body to inhibit fluid flow through the second port. The valve also includes at least one projection extending radially inwardly from an inner surface of the tube. The projection extends into the lumen through an aperture defined by the catheter body. The projection is configured to be displaced in a radially outward direction by a deliverable instrument received by the lumen, thereby pushing a portion of the tube away from the outer surface of the catheter body to a second position to permit fluid flow through the second port. The aperture typically is the second port. The tube has first and second ends, the first end being disposed distally of the second end on the catheter body and secured to the catheter body.

In yet another aspect, a catheter includes a longitudinally extending catheter body having proximal and distal ends. The catheter body defines a lumen extending longitudinally from a first port, defined at the proximal end, through the body to a second port defined at the distal end. The catheter includes a self-sealing fluid barrier disposed in the lumen for controlling fluid flow through the second port. The self-sealing fluid barrier is configured to be pierced a deliverable instrument. The self-sealing fluid barrier extends across the lumen. The received deliverable instrument defines a lumen therethrough that allows fluid flow.

In another aspect, a catheter includes a longitudinally extending catheter body having proximal and distal ends. The catheter body defines a lumen extending longitudinally from a first port, defined at the proximal end, through the body to a second port defined at the distal end. The catheter includes a valve for controlling fluid flow through the second port. The valve includes first and second compliant bladders mounted opposite each other in the lumen and disposed in a first position in sealing engagement to inhibit fluid flow through the second port. The bladders are configured to receive a deliverable instrument therebetween. The received deliverable instrument defines a lumen therethrough that allows fluid flow.

In another aspect, a catheter includes a longitudinally extending catheter body having proximal and distal ends. The catheter body defines a lumen extending longitudinally from a first port, defined at the proximal end, through the body to a second port defined at the distal end. The catheter includes a means for selectively controlling fluid flow through the second port.

In another aspect, a catheter system includes a catheter and a catheter primer. The catheter includes a longitudinally extending catheter body having proximal and distal ends. The catheter body defines a lumen extending longitudinally from a first port, defined at the proximal end, through the body to a second port defined at the distal end. The catheter includes a valve for controlling fluid flow through the second port. The valve includes a valve element disposed within the lumen and a valve element operator defined by the valve element. The valve element transitions between a first state in sealing engagement with a valve seat defined by the catheter body to inhibit fluid flow through the second port, and a second state in which the valve element is spaced from the valve seat to permit fluid flow through the second port. The valve element operator is configured for engagement by a deliverable instrument received by the lumen to cause a transition of the valve element between the first state to the second state. The catheter primer includes a barrel having open proximal and distal ends and a plunger receivable in the barrel and movable therealong. The catheter primer also includes a shank attached to the plunger and configured to actuate the plunger. A handle is secured to the barrel, and a spring biases the shank toward the distal end of the barrel.

Implementations of this aspect of the disclosure may include one or more of the following features. In some implementations, the catheter primer includes a spring retainer coupled to the proximal end of the barrel and configured to retain the spring. The valve element, in some examples, is secured in the lumen to the catheter body and extends circumferentially along a wall of the lumen over the second port to a distal tip. The valve element is torsionally biased against the lumen wall in sealing engagement with the valve seat defined by the lumen wall. Circumvolution of the valve element operator disposed at the distal tip of the valve element causes the valve element to elastically deform from the first position to the second position for permitting fluid flow through the second port. A distal tip of the received deliverable instrument is configured to engage the valve element operator, such that torsional movement of the received deliverable instrument is translated to the valve element operator.

In some implementations, the valve seat is an orifice defined by the catheter body in the lumen, and the valve element is disposed distally of the orifice and spring biased toward sealing engagement with the valve seat to occlude the orifice. The valve element operator extends through the orifice for actuation of the valve by the received deliverable instrument.

In some examples, the valve element is a lumen plug slidably disposed within the lumen for movement between a first position that permits fluid flow through the second port, and a second position in sealing engagement with the valve seat defined by a wall of the lumen, occluding the lumen. The lumen plug defines a fluid channel extending from a longitudinal proximal end of the lumen plug to a lateral side of the lumen plug. The fluid channel is in fluid communication with the second port when the lumen plug is in the first position. The lumen plug is biased by a spring toward sealing engagement with the valve seat.

The valve element, in some examples, is a gate pivotally attached to the catheter body in the lumen. The gate swings from the first position, in sealing engagement with the valve seat, to the second position when moved by the received deliverable instrument. A side wall of the catheter body may define the second port. The valve element extends along a wall of the lumen for sealing engagement with the valve seat defined by the lumen wall. The valve element operator extends at least partially across the lumen for actuation by the received deliverable instrument.

In some implementations, the valve element is disposed in the lumen and is elastically deformed by the deliverable instrument when moved from the first position, in sealing engagement with the valve seat, to the second position for permitting fluid flow through the second port.

In some examples, the valve element is an ovular tube having inner and outer walls. A portion of the inner wall of the tube defines the valve element operator. The received deliverable instrument engages the valve element operator to elastically alter a geometry of the tube to the second position for permitting fluid flow though the second port.

The valve element, in some examples, is an inflatable bladder disposed in the lumen and operable to distend between a first position in sealing engagement with the valve seat and the second position for allowing fluid flow through the second port. The catheter body defines a bladder inflation channel in fluid communication with the bladder to control bladder inflation.

In some implementations, the catheter body defines at least one axial groove in a wall of the lumen. The groove extends from the proximal end of the catheter body to the distal end of the catheter body. The catheter body may also, or instead, define at least one axial protrusion in a wall of the lumen. The protrusion extends from the proximal end of the catheter body to the distal end of the catheter body.

In another aspect, a catheter includes a longitudinally extending catheter body having proximal and distal ends. The catheter body defines a lumen extending longitudinally from a first port, defined at the proximal end, through the body to a second port, defined at the distal end. A distal portion of the body defines a valve for controlling fluid flow through the second port. The distal portion of the body defines a substantially U-shaped cross-section having first and second substantially U-shaped flaps joined along lateral edges. The flaps move between a first state permitting fluid flow through the second port and a second state in sealing engagement with each other when pressure in the lumen drops below a threshold pressure.

Implementations of this aspect of the disclosure may include one or more of the following features. In some implementations, the catheter includes a guide wire tube disposed over the body and positioned to define a guide wire opening with a transition section of the body in between the valve and a main section of the body. The catheter may include a reinforcing member embedded in the body at least along the distal portion of the body. Examples of the reinforcing member include a rail disposed substantially along the lateral edges of the flaps, a coil, and/or braids. Other suitable structures may be used as reinforcing members as well or in conjunction with the aforementioned examples.

The details of one or more implementations of the disclosure are set forth in the accompanying drawings and the description below. Other features, objects, and advantages will be apparent from the description and drawings, and from the claims.

### DESCRIPTION OF DRAWINGS

FIG. 1 is a side sectional view of a catheter having a valve that controls fluid flow through a lumen defined by the catheter.

FIG. 2 is a side sectional view of a distal end of a catheter having a valve including a compliant flap.

FIG. 3A is a side sectional view of a distal end of a catheter having a valve including at least two compliant flaps.

FIG. 3B illustrates an example of a deliverable instrument actuating the flaps of the valve shown in FIG. 3A.

FIG. 4A is a perspective view of a catheter with a valve formed by a distal portion of its catheter body.

FIG. 4B is a section view of the catheter shown in FIG. 4A.

FIG. 4C is a perspective view of the catheter shown in FIG. 4A with a guide wire tube disposed over the catheter body.

FIG. 4D is a perspective view of a catheter with a valve formed by a distal portion of its catheter body and reinforced by an embedded support rail.

FIG. 4E is a perspective view of a catheter with a valve formed by a distal portion of its catheter body and reinforced by an embedded coil.

FIG. 4F is a perspective view of a catheter with a valve formed by a distal portion of its catheter body and reinforced by embedded braids.

FIG. 5 is an end view of a deliverable instrument with a grooved outer surface.

FIG. 6 is a side sectional view of a distal end of a catheter having a valve including an elastic tube that surrounds a portion of the catheter body.

FIG. 7 is a side sectional view of a distal end of a catheter having a valve including a spring that biases a valve element into sealing engagement with a valve seat.

FIG. 8 is a side sectional view of a distal end of a catheter having a valve that includes two compliant bladders opposedly mounted in a lumen and disposed in sealing engagement with each other.

FIG. 9 is a side sectional view of a distal end of a catheter having a valve that includes a membrane disposed across a lumen.

FIG. 10 is a cross-section view at line 10-10 of the catheter shown in FIG. 9.

FIG. 11 is a cross-section view at line 11-11 of the catheter shown in FIG. 9, illustrating a longitudinal channel and protrusion defined by the catheter body in the lumen.

FIG. 12 is a side sectional view of a distal end of a catheter having a valve including an elastomeric tubular membrane disposed in a guide-wire lumen in a first position to inhibit fluid flow through a second port defined by the catheter.

FIG. 13 is a side sectional view of a distal end of a catheter having a filter disposed in a lumen.

FIG. 14 is a side sectional view of a deliverable instrument in a lumen of a catheter as it actuates a valve that controls fluid flow through the lumen.

FIG. 15 is a side sectional view of a distal end of a catheter having a valve sealed by a valve element torsionally biased against a lumen wall.

FIG. 16 is across-section view at line 16-16 of the catheter shown in FIG. 15.

FIG. 17 is a side sectional view of a deliverable instrument in a lumen of a catheter as it actuates a valve that controls fluid flow through the lumen.

FIG. 18 is a side sectional view of a distal end of a catheter having a valve configured as a lumen plug for sliding along through a lumen to control fluid flow through a second port.

FIG. 19 is across-section view at line 19-19 of the catheter shown in FIG. 18.

FIG. 20 is a side sectional view of a distal end of a catheter having a valve in which a spring-biased lumen plug slides through a lumen to control fluid flow through a second port.

FIG. 21 is a side sectional view of a distal end of a catheter having a valve including a valve element pivotally attached to the catheter body in a lumen.

FIG. 22 is a side sectional view of a distal end of a catheter having a valve including a compliant valve element attached to the catheter body in a lumen.

FIG. 23 is a side sectional view of a distal end of a catheter having a valve including a compliant tubular valve element controlling fluid flow through a second port.

FIG. 24 is a cross-section view at line 24-24 of the catheter shown in FIG. 23.

FIG. 25 is a side sectional view of a distal end of a catheter having a valve including a valve element configured as an inflatable bladder disposed in a lumen.

FIG. 26 is a side sectional view of a deliverable instrument inserted into a catheter and actuating a valve that controls fluid flow through a second port defined by the catheter.

FIG. 27 is a side sectional view of a distal end of a catheter having a valve including an elastic tube surrounding a portion of the catheter for selectively sealing a second port defined by the catheter.

FIG. 28 is a side sectional view of a catheter having a self-sealing fluid barrier disposed in a lumen.

FIG. 29 is a side sectional view of the distal end of the catheter shown in FIG. 28 with a deliverable instrument piercing the self-sealing fluid barrier.

FIG. 30 is a side sectional view of a catheter having two opposedly mounted bladders disposed in sealing engagement in a lumen to control fluid flow through a second port defined by the catheter.

FIG. 31 is a side sectional view of the distal end of the catheter shown in FIG. 30 with a deliverable instrument inserted between the two opposedly mounted bladders.

FIG. 32 is a side sectional view of catheter system having a catheter and a catheter primer.

FIG. 33 is a side sectional view of a catheter primer.

Like reference symbols in the various drawings indicate like elements.

### DETAILED DESCRIPTION

This disclosure presents priming concepts and the use thereof on or integrated with catheters (e.g. rapid exchange type catheters). A primable catheter allows, inter alia, bench top saline priming of an inner lumen while preventing backflow of blood while in a body with a guide wire in place. Some catheters require priming to achieve a bubble-free inner fluid in its lumen for proper functioning and use.

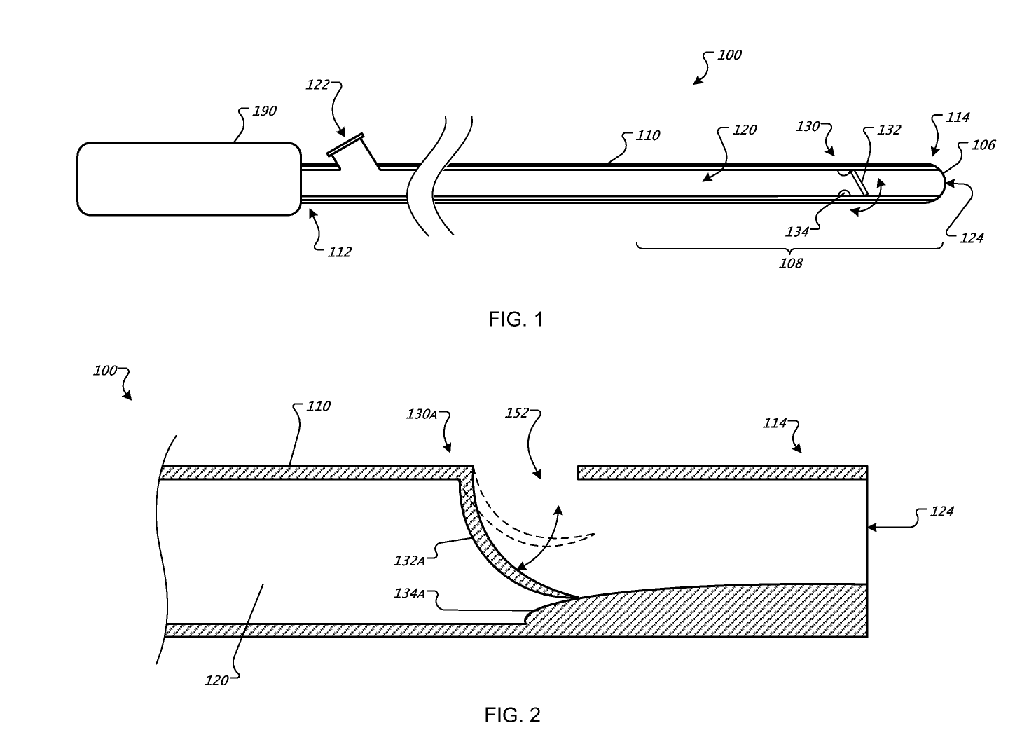

Referring to FIG. 1, a catheter 100 includes a longitudinally extending body 110 having proximal and distal ends, 112 and 114 respectively, and defining at least one working lumen 120. A handle or hub 190 may be disposed at the proximal end 112 for holding and manipulating the catheter 100. The working lumen 120 extends longitudinally from a first port 122 defined at the proximal end 112 through the body 110 to a second port 124 defined at the distal end 114. A valve 130 in the working lumen 120 is in fluid communication with the second port 124, controlling fluid flow through the second port 124 by selectively interrupting fluid communication between the second port 124 and the working lumen 120.

In some implementations, the catheter 100 is 3.2 French (F) in diameter and 165 cm in working length (e.g. the portion used for insertion into a patient). The catheter body 110 may including a soft, atraumatic tip 106 at the distal end 114, a clear imaging window 108, and a relatively stiffer proximal shaft or body 110. A shapeable nitinol wire may be embedded in the distal tip 106 to facilitate access in tortuous anatomy and stabilize the tip 106 while the catheter 100 is being introduced into a target artery.

In one implementation, the valve 130 includes a valve element 132 pivotally attached to the catheter body 110 and extending into the working lumen 120. A valve seat 134 defined by the catheter body 110 in the working lumen 120 receives the valve element 132. The valve element 132 is disposed to move between a first state in which it forms a seal at the valve seat 134 and a second state spaced from the valve seat 134 for permitting fluid flow through the second port 124. A transition between the first and second states occurs when a hydrostatic pressure in the working lumen 120 exceeds a threshold pressure. In some examples, the valve element 132 is biased toward the first state in sealing engagement with the valve seat 134.

In some implementations, as shown in FIG. 2, the catheter 100 includes a valve 130A having a compliant flap 132A secured to the catheter body 110 in the working lumen 120 and extending across the working lumen 120. A valve seat 134A defined by the catheter body 110 in the working lumen 120 receives the flap 132A. In the absence of fluid pressure, the flap 132A assumes a first configuration in sealing engagement with the valve seat 134A. The flap 132A elastically deflects to a second configuration spaced from the valve seat 134A for permitting fluid flow through the second port 124 when a hydrostatic pressure in the working lumen 120 exceeds a threshold pressure. In the example shown, the catheter body 110 defines a guide wire port 152 adjacent to the valve 130A and in fluid communication with the working lumen 120.

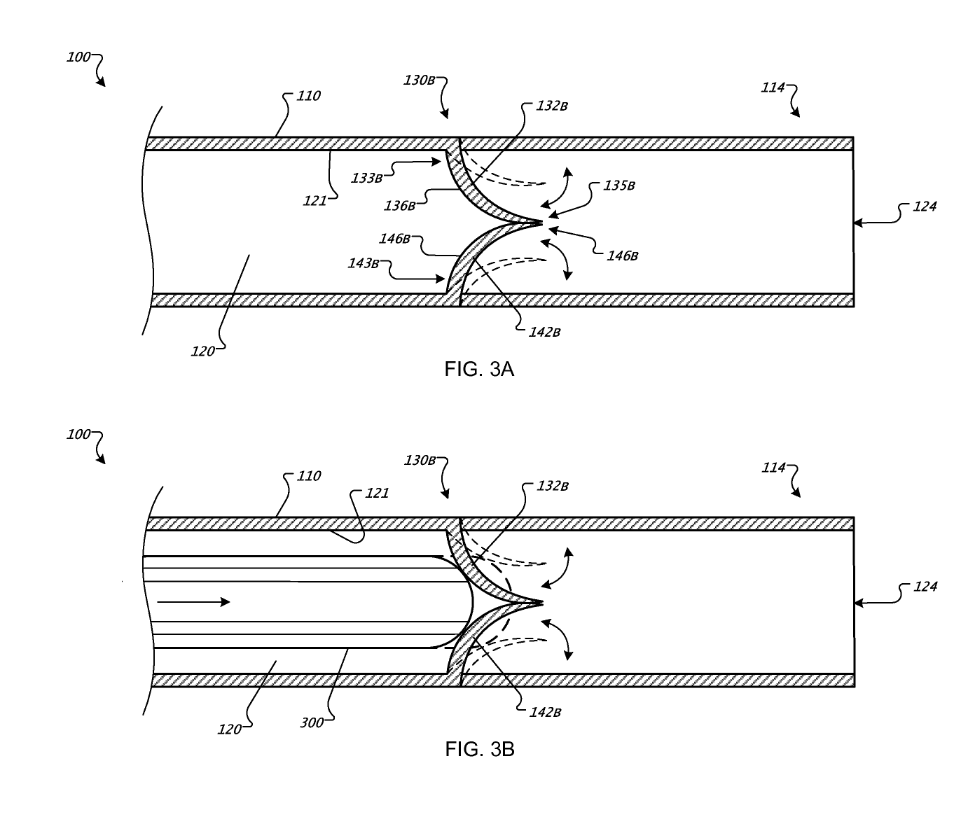

In some implementations, as shown in FIG. 3A, the catheter 100 includes a valve 130B having at least first and second compliant flaps 132B and 142B, respectively, secured opposite each other to an inner wall 121 of the working lumen 120. In some examples, the valve 130B includes three or more compliant flaps (e.g. tricuspid configuration). The flaps 132B, 142B elastically deform to transition between a first state in which the flaps 132B, 142B are in sealing engagement with each other, thereby inhibiting fluid flow through the second port 124, and a second state, in which the flaps 132B, 142B allow fluid flow through the second port 124 and out of the working lumen 120. A transition between the first and second states occurs when a hydrostatic pressure in the working lumen 120 exceeds a threshold pressure.

The first and second compliant flaps 132B, 142B each have proximal ends 133B, 143B and distal ends 135B, 145B with a broad side 136B, 146B extending therebetween. The proximal ends 133B, 143B of the flaps 132B, 142B are secured along the circumference of the inner wall 121 of the working lumen 120. In the first state, the flaps 132B, 142B extend toward each other and meet along the broad sides 136B, 146B to temporarily inhibit fluid flow through the second port 124.

FIG. 3B illustrates an example of a deliverable instrument 300 causing the flaps 132B, 142B to transition between their first and second states. In this case, inner lumen pressure plays only a minor role in actuating the flaps 132B, 142B.

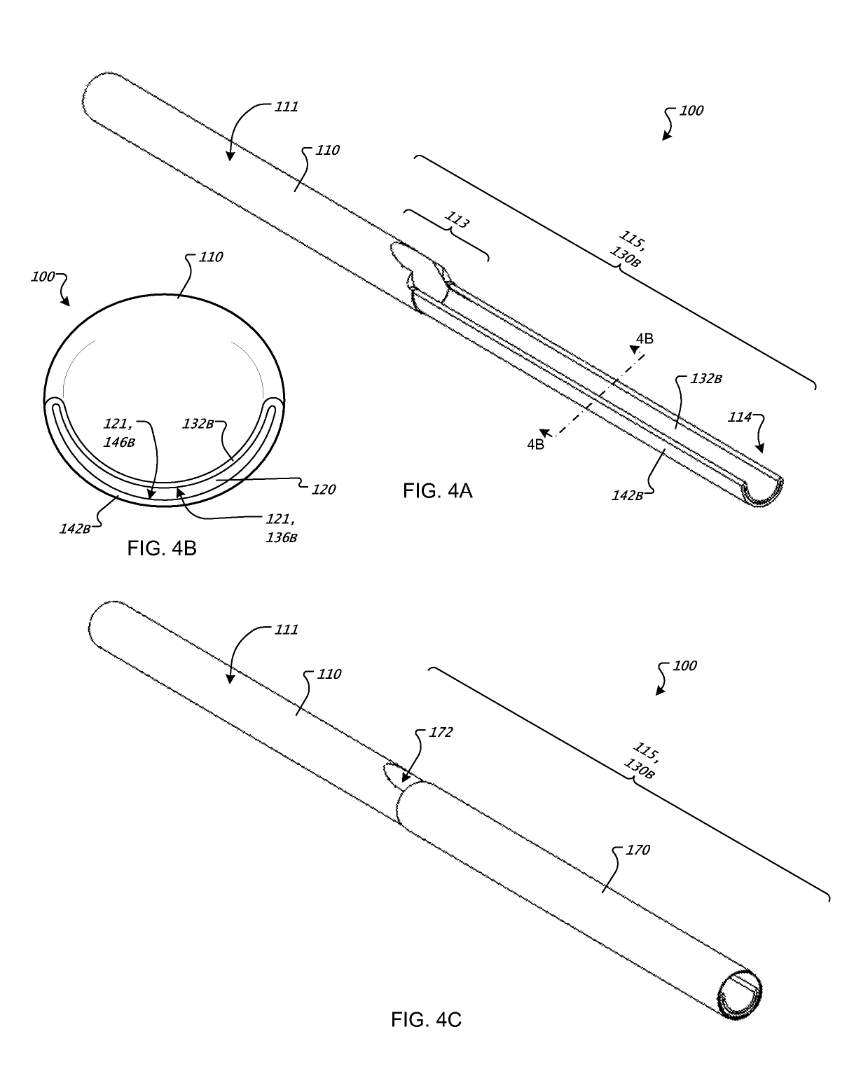

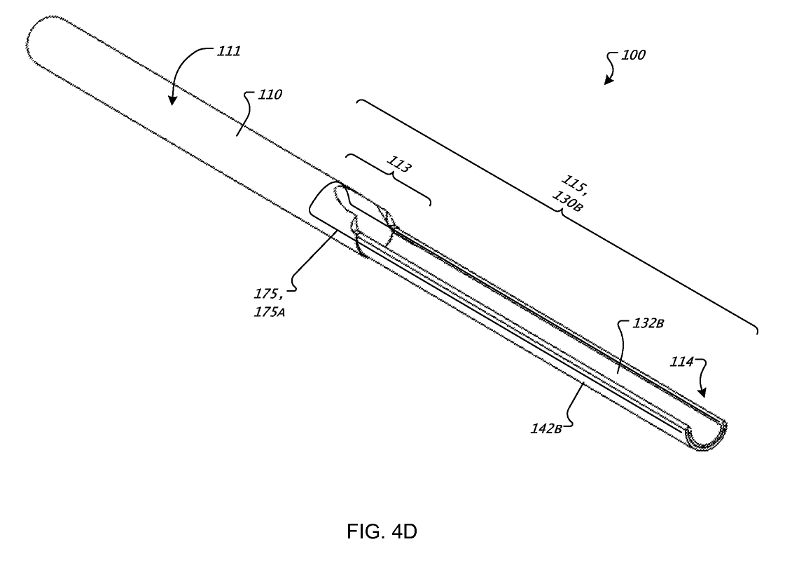

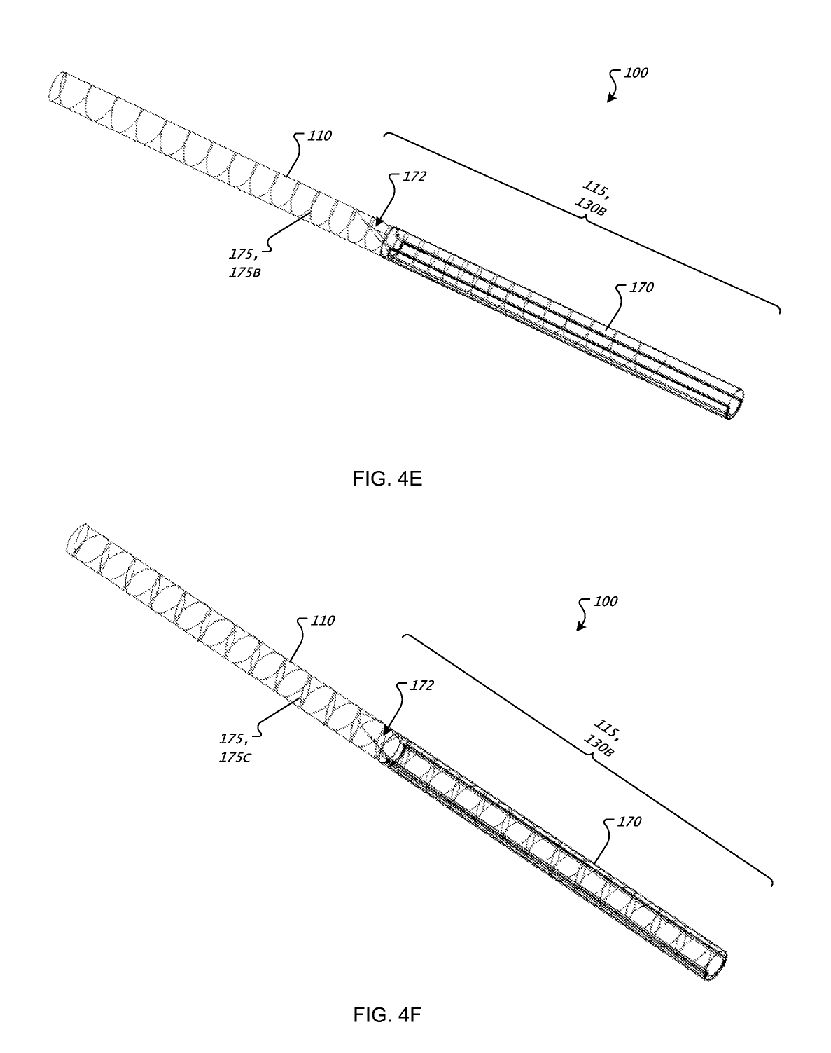

In the example illustrated in FIGS. 4A-4C, the catheter 100 includes a valve 130B formed by a thinned out distal portion 115 of the catheter body 110 pressed into a substantially “U” shaped cross-section and deformed into first and second compliant flaps 132B and 142B joined along lateral edges, to remain a tube. The distal portion 115 of the catheter body 110 is stretched and thermally and/or mechanically deformed (e.g. axial pleated) such that its cross section becomes substantially “U” shaped for about 2 cm. About half of the external surface 111 of the distal portion 115 of the catheter body 110, which was deformed, forms an inner portion 111A of the “U” shaped channel that acts as a trough for a guide wire. The inner wall 121 of the working lumen 120 defines first and second broad sides 136B, 146B of the respective flaps 132B, 142B concomitantly juxtaposed (e.g. substantially face to face). The valve 130B permits fluid flow through the working lumen 120 under positive inner lumen pressure. Negative inner lumen pressure causes closure of the valve 130B by pulling the first and second broad sides 136B, 146B of the respective flaps 132B, 142B into sealing contact with each other, thus preventing fluid flow into the distal end 114 of the catheter 100.

Referring to FIG. 4C, in some examples, a guide wire tube 170 (e.g. approximately 2 cm long) is positioned over the distal portion 115 of the catheter body 110 that forms the valve 130B and secured to the catheter body 110 in a position that provides a sufficient guide wire opening 172 for a guide wire to pass over the distal portion 115 of the catheter 100 while passing through the guide wire tube 170. A transition 113 between the “U” shaped channel 115 and the normal (e.g. round) catheter body 110 should be sufficiently long to avoid stresses of the guide wire and the sheath. This transition section 113 may become further stressed as it tracks the indwelling guide wire around substantial curves. Priming of the catheter 100 can occur on a bench prior to use. The priming is performed without a guide wire in the guide wire tube 170, thus allowing the flaps 132B, 142B to move and/or the distal portion 115 of the catheter body 110 to distend, opening the valve 130B. Placement of a guide wire in the guide wire tube 170 over the distal portion 115 of the catheter body 110 at least partially closes the valve 130B (preferably the valve 130B is fully closed).

A reinforcement member 175 may be used to increase mechanical support for the distal portion 115 of the catheter body 110. The reinforcement member 175 can be embedded in the distal portion 115 of the catheter body 110 to prevent the walls of the distal portion 115 from breaking (e.g. bowing out or ripping). In some examples, the reinforcement member 175 is a support rail 175A disposed within the walls of the distal portion 115 of the catheter body 110. The support rail 175A runs from the distal end 114 to the transition section 113, where the support rail 175A bends laterally and into an arch or complete circle before returning to the distal end 114, as shown in FIG. 4D. The support rail 175 comprises stainless steel wire or Nitinol wire, in some examples, having a diameter of approximately 0.002″. In other examples, the reinforcement member 175 is a coil 175B disposed within the walls of the distal portion 115 of the catheter body 110, as shown in FIG. 4E. The coil 175B spans the entire length of the distal portion 115 and continues proximally over the transition section 113 to a comfortable position (e.g. 2 cm past the transition section 113) along the unmodified catheter body 110, with an aperture 152 for the guide wire. The coil 175B may be imbedded into the catheter body 110 or adhered to its surface 111. Further reinforcement includes forming the catheter body 110 from an extrusion having axial ribs. In some examples, the reinforcement member 175 includes braids 175C embedded in the catheter body 110, as shown in FIG. 4F.

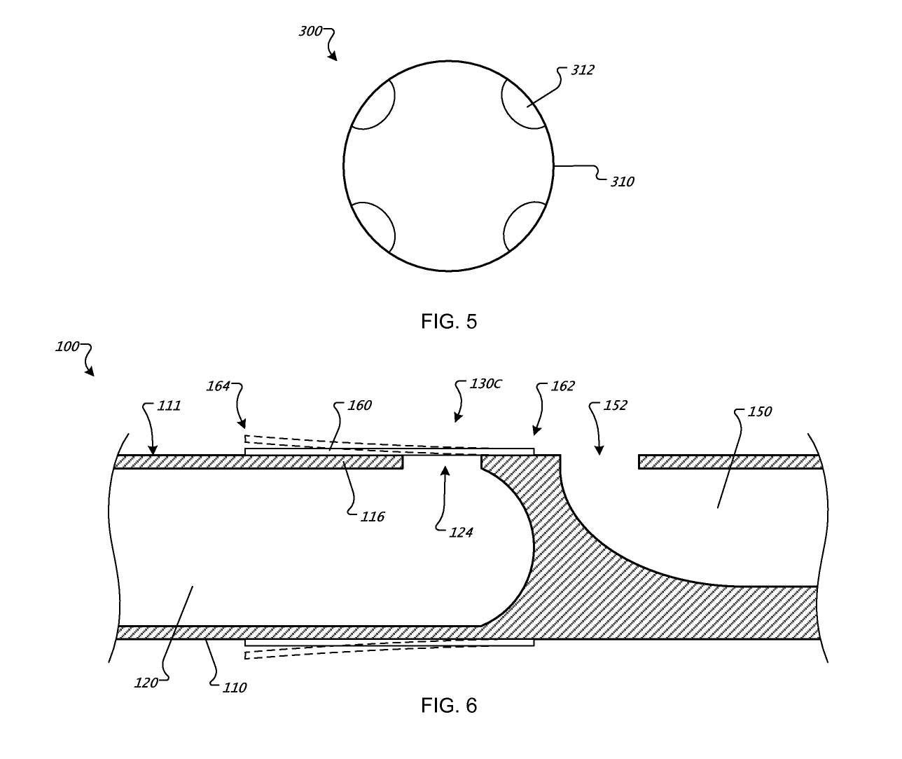

In some instances, an outer surface 310 of the deliverable instrument 300 defines channels or grooves 312, as shown in FIG. 5. The grooves 312 allow fluid to flow therethrough when the deliverable instrument 300 penetrates past the flaps 132B, 142B. The fluid escaping through the grooves 312 then escapes through the second port 124.

In the embodiment illustrated in FIG. 6, the catheter 100 includes a valve 130C having an elastic tube 160 surrounding the catheter body 110. The tube 160 rests in a first state in sealing engagement with an outer surface 111 of the catheter body 110 to temporarily seal the second port 124. When hydrostatic pressure in the working lumen 120 exceeds a threshold pressure, the tube 160 distends to a second state that allows fluid flow out of the second port 124. A first end 162 of the tube 160 is disposed distally of a second end 164 on the catheter body 110. The first end 162 is bonded, heat sealed, ultrasonically welded, or otherwise attached to the catheter body 110.

The catheter 100 may be intended for introduction into a patient's vasculature via a 0.014 inch coronary guide wire. In the example shown, the catheter body 110 defines a guide wire port 152 adjacent to the valve 130C and in fluid communication with a guide wire lumen 150. The guide wire lumen 150 allows the catheter 100 to track along a guide wire (not shown). The guide wire may be allowed to exit approximately 25 mm proximal to the distal tip 106. The working lumen 120 terminates at the port 124 defined, in this case, in the catheter body sidewall 116. As a result, the working lumen 120 is isolated from the guide wire lumen 150. In some implementations, the tube 160 is non-elastic and moves between open and closed positions over the port 124 by sliding or pivoting over the catheter body 110.

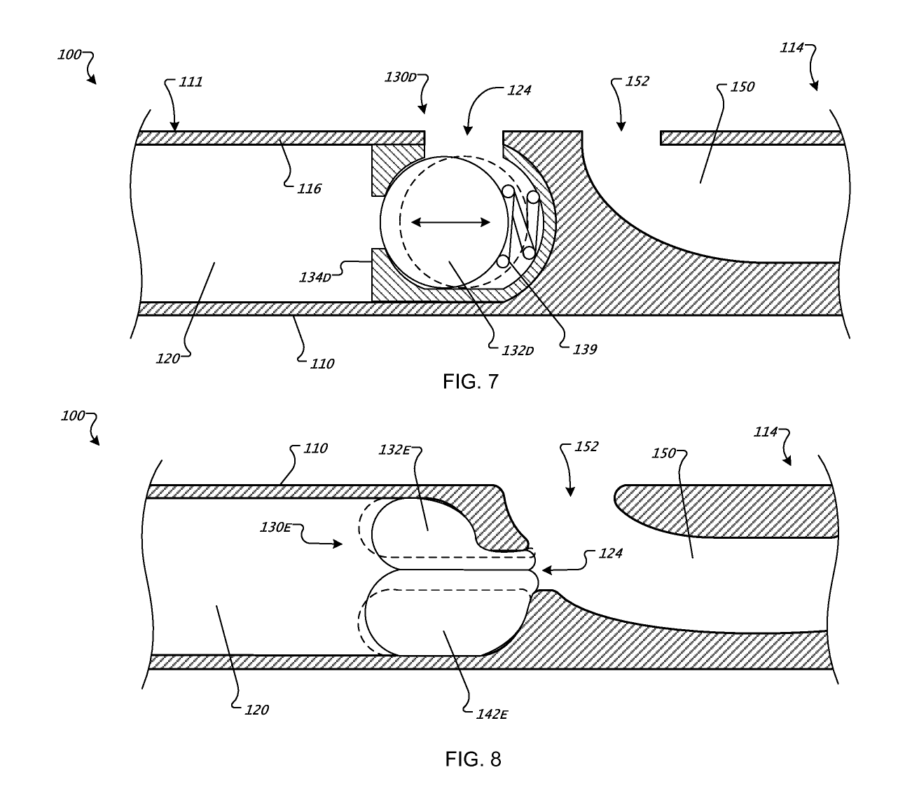

Referring to FIG. 7, in some implementations, the catheter 100 includes a valve 130D having a movable valve element 132D disposed in the working lumen 120 and a valve seat 134D defined by the catheter body 110 in the working lumen 120. A spring 139 biases the valve element 132D toward a first position in sealing engagement with the valve seat 134D. The valve element 132D is operable to move from the first position in sealing engagement with the valve seat 134D to a second position spaced from the valve seat 134D for permitting fluid flow through the second port 124. In the example shown, the second port 124 is defined in the catheter body sidewall 116. The valve element 132D moves from the first position to the second position when a hydrostatic pressure in the working lumen 120 exceeds a threshold pressure.

Referring to FIG. 8, in some implementations, the catheter 100 includes a valve 130E having first and second compliant bladders 132E, 142E mounted opposite each other in the working lumen 120. In a first position, the bladders 132E, 142E are in sealing engagement with each other to inhibit fluid flow through the second port 120. In a second position, the bladders 132E, 142 are spaced from each other and therefore permit fluid flow through the second port 124. The bladders 132E, 142E transition from the first position to the second position when a hydrostatic pressure in the lumen exceeds a threshold pressure. The bladders 132E, 142E may be inflated with a gas (e.g. air) or filled with a compressible foam or gel. Therefore, when the lumen pressure exceeds the threshold pressure, fluid flows between the two bladders 132E, 142E, through the second port 124, and into the guide wire lumen 150. In the example shown, the catheter body 110 defines a guide wire port 152 adjacent to the valve 130C and in fluid communication with a guide wire lumen 150.

In some implementations, one inserts the deliverable instrument 300 shown in FIG. 5 between the two bladders 132E, 142E. As discussed with reference to FIG. 4, the grooves 312 defined by the outer surface 310 of the deliverable instrument 300 allow fluid to flow therethrough when the deliverable instrument 300 penetrates past the two bladders 132E, 142E. The fluid escaping through the grooves 312 then exits the second port 124 and enters the guide wire lumen 150.

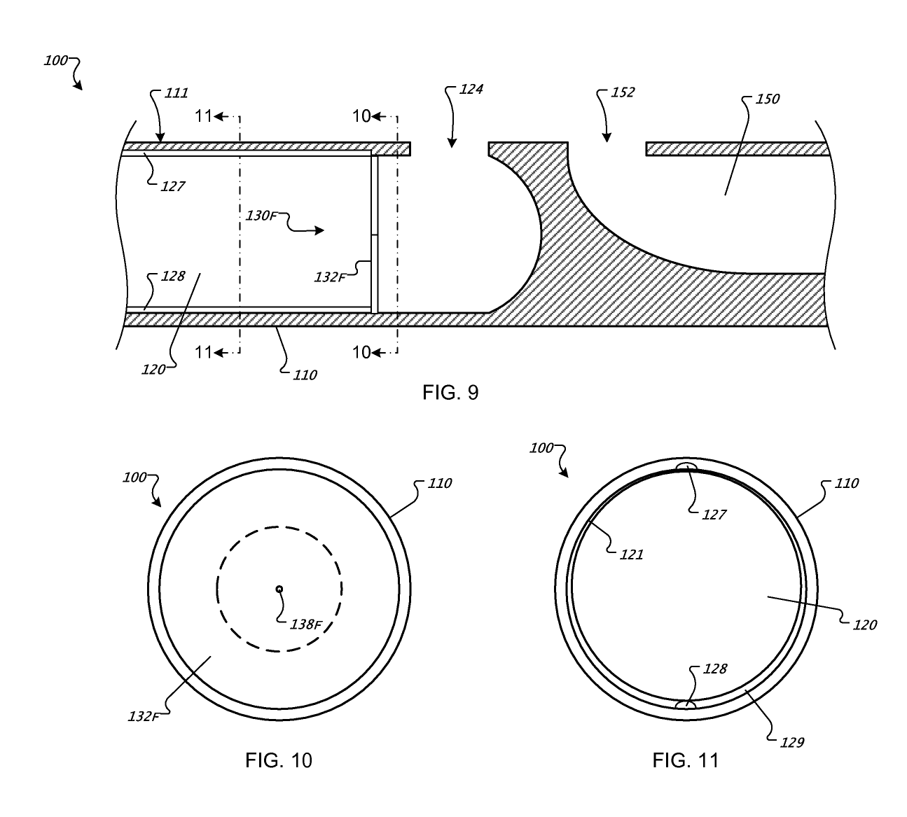

In the examples illustrated in FIGS. 9-10, the catheter 100 includes a valve 130F having a membrane 132F disposed across the working lumen 120. The membrane 132F defines an aperture 138F (see FIG. 10) tending to remain closed to inhibit fluid flow through the second port 124. The aperture 138F opens for permitting fluid flow through the second port 124 when a hydrostatic pressure in the working lumen 120 exceeds a threshold pressure.

In some examples, as shown in FIG. 11, the catheter body 110 defines at least one axial groove 127 in a wall 121 of the working lumen 120. The groove 127, which extends from the proximal end 112 of the catheter body 110 to the distal end 114 of the catheter body 110, provides a pathway for fluid to flow when the deliverable instrument 300 is inserted in the working lumen 120. In some cases, the catheter body 110 defines at least one axial protrusion 128 in a wall 121 of the working lumen 120. The protrusion 128 may extend from the proximal end 112 of the catheter body 110 to the distal end 114 of the catheter body 110. The protrusion 128 creates a gap 129 between the deliverable instrument 300 and the inner wall 121 of the working lumen 120. Fluid is allowed to flow through the gap 129, for example, to provide flushing of the working lumen 120. As a result, the groove 127 and the protrusion, either individually or in combination, thereby prevent adverse pressure effects, such as a suction effect, when the deliverable instrument 300 is removed from the working lumen 120.

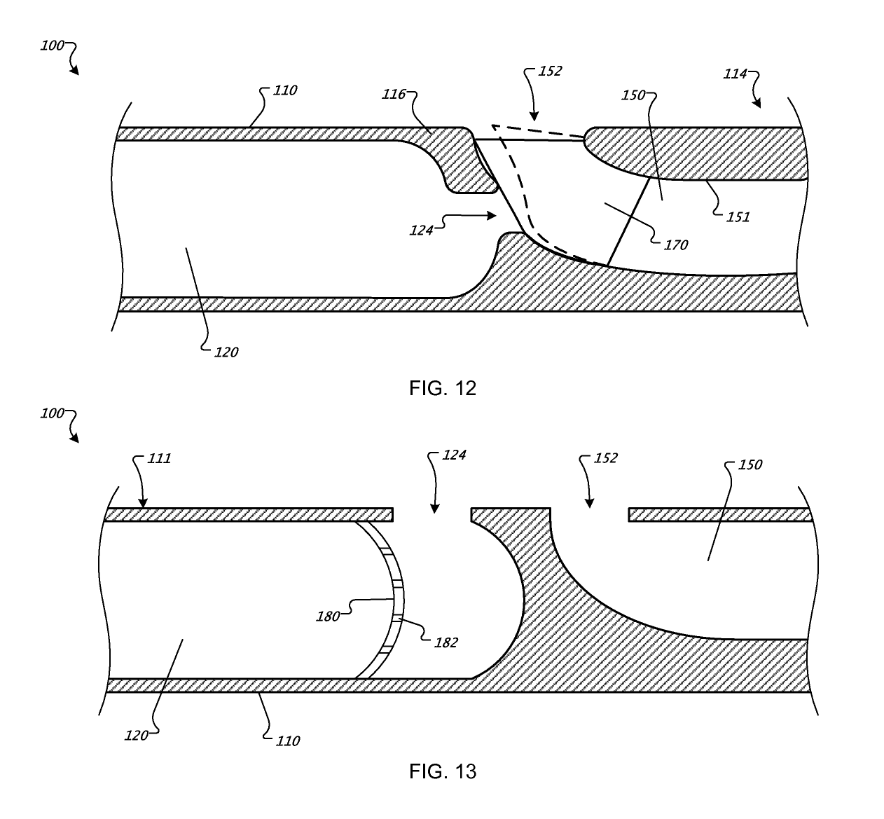

Referring to FIG. 12, in some implementations, the catheter 100 includes a longitudinally extending catheter body 110 having proximal and distal ends, 112 and 114 respectively. The catheter body 110 defines a first lumen 120 (e.g. working lumen) extending longitudinally from a first port 122 (see e.g. FIG. 1) defined at the proximal end 112 (see e.g. FIG. 1) through the catheter body 110 to a second port 124 defined at the distal end 114. The catheter body 110 defines a second lumen 150 (e.g. guide wire lumen) extending from a side port 152 (e.g. a guide wire port) defined in a side 116 of the catheter body 110 to the distal end 114 of the catheter body 110. The second port 124 joins the first and second lumens 120, 150. An elastomeric tubular membrane 170 (e.g. hose) is disposed in the second lumen 150 in a first state in sealing engagement with a wall 151 of the second lumen 150. In this first state, the membrane 170 inhibits fluid flow through the second port 124. The membrane 170 elastically deflects to a second state spaced from the wall 151 of the second lumen 150 when a hydrostatic pressure in the first working lumen 120 exceeds a threshold pressure. In this second state, the membrane 170 permits fluid to flow through the second port 124 into the second lumen 150. This fluid exits through the side port 152 of another distal port (e.g. a guide wire port) in fluid communication with the second lumen 150. In some examples, the tubular membrane 170 adheres to the wall 151 of the second lumen 150, preferably past the second port 124. In other examples, retaining elements (e.g. protrusions) disposed on, or defined by, the wall 151 of the second lumen 150 hold the tubular membrane 170 in place.

In the example illustrated in FIG. 13, the catheter 100 includes a longitudinally extending catheter body 110 having proximal and distal ends, 112 and 114 respectively. The catheter body 110 defines a first working lumen 120 extending longitudinally from a first port 122 (see e.g. FIG. 1) defined at the proximal end 112 (see e.g. FIG. 1) through the catheter body 110 to a second port 124 defined at the distal end 114. A filter 180, disposed in the working lumen 120 in fluid communication with the second port 124, controls fluid flow through the second port 124 prevents blood flow therethrough.

In some examples, the filter 180 defines fluid passageways 182 therethrough having diameters between about 8 microns and about 100 microns. The fluid passageways 182 may be tortuously defined (e.g. to be winding or meandering), so as to create an impedance for fluid flow.

In some implementations, the filter 180 includes an open cell foam. During priming of the catheter 100 with a fluid (e.g. saline), the open cell foam filter 180 absorbs the fluid and eventually clogs the working lumen 120 at the filter 180 upon saturation. Examples of open cell foams include: Polyester, Polyether Polyurethane, Low Permeability, Polyimide, and Melamine. Once clogged, the filter 180 impedes any further fluid flow through the second port 124, effectively closing the second port 124.

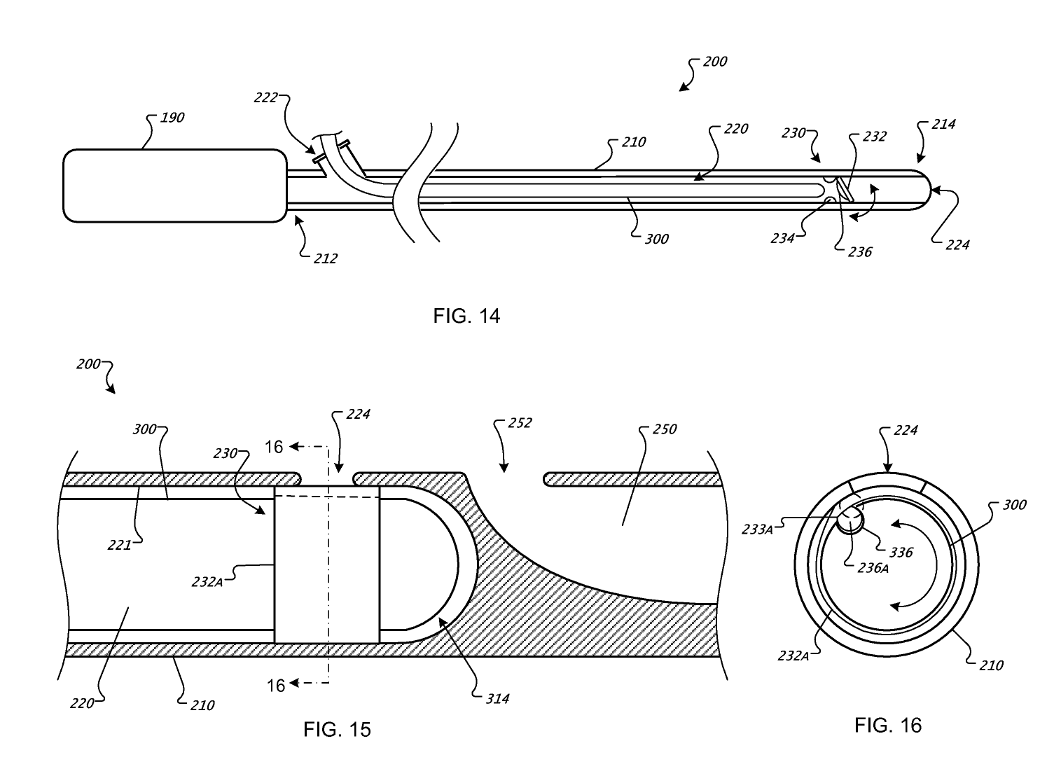

In the example illustrated in FIG. 14, a catheter 200 includes a longitudinally extending catheter body 210 having proximal and distal ends, 212 and 214 respectively. The catheter body 210 defines a working lumen 220 extending longitudinally from a first port 222 defined at the proximal end 212 through the catheter body 210 to a second port 224 defined at the distal end 214. A handle 190 is disposed at the proximal end 112 for holding and manipulating the catheter 100. A valve 230 in fluid communication with the second port 224 controls fluid flow through the second port 224. The valve 230 includes a valve element 232 disposed within the working lumen 220 for transition between a first state, in which the valve element 232 is in sealing engagement with a valve seat 234 defined by the catheter body 210 and thereby inhibits fluid flow through the second port 224, and a second state, in which the valve element 232 is spaced from the valve seat 234 and thereby permits fluid flow through the second port 234. A deliverable instrument 300 inserted in the working lumen 220 engages a valve element operator 236 defined by the valve element 232 to cause movement of the valve element 232 from the first state to the second state, permitting fluid flow through the second port 224.

The catheter 100 may be used as a spectroscopic coronary catheter, in which case the deliverable instrument 300 may include a torque transmission cable with a rotating hub and an optical imaging core. The torque transmission cable may be composed of a hi-torque, flexible rotating drive cable. In some examples, the optical imaging core includes two optical fibers bonded at their distal ends to an outward looking micro-optical tip. Pullback and rotation of the torque cable imparts pullback and rotation to the optical tip, causing the optical tip to move within the optically transparent transmission window portion of the sheath, thus scanning the artery.

Referring to FIGS. 15-16, in some implementations, the catheter 100 includes a valve 230A having a valve element 232A secured in the working lumen 220 to the catheter body 220 and extending circumferentially along a wall 221 of the working lumen 220 over the second port 224 to a distal tip 233A. The valve element 232A is torsionally biased into a first position against the lumen wall 221 in sealing engagement with the second port 224 defined by the lumen wall 221. Circumvolution of the valve element operator 236A (see FIG. 16) disposed at the distal tip 233A of the valve element 232A causes the valve element 232A to elastically deform from the first position to a second position, in which it permits fluid flow through the second port 224. A distal tip 314 of the received deliverable instrument 300 engages the valve element operator 236A, such that torsional movement of the received deliverable instrument 300 applies a torsional force to the valve element operator 236A. In the example shown, the distal tip 314 of the received deliverable instrument 300 defines a recess 336 configured to receive the valve element operator 236A (e.g. a ball shaped protrusion). In other examples, the distal tip 314 of the received deliverable instrument 300 is magnetically secured to the valve element operator 236A (e.g. via a magnet in the distal tip 314 and a magnetically attractable valve element operator 236A).

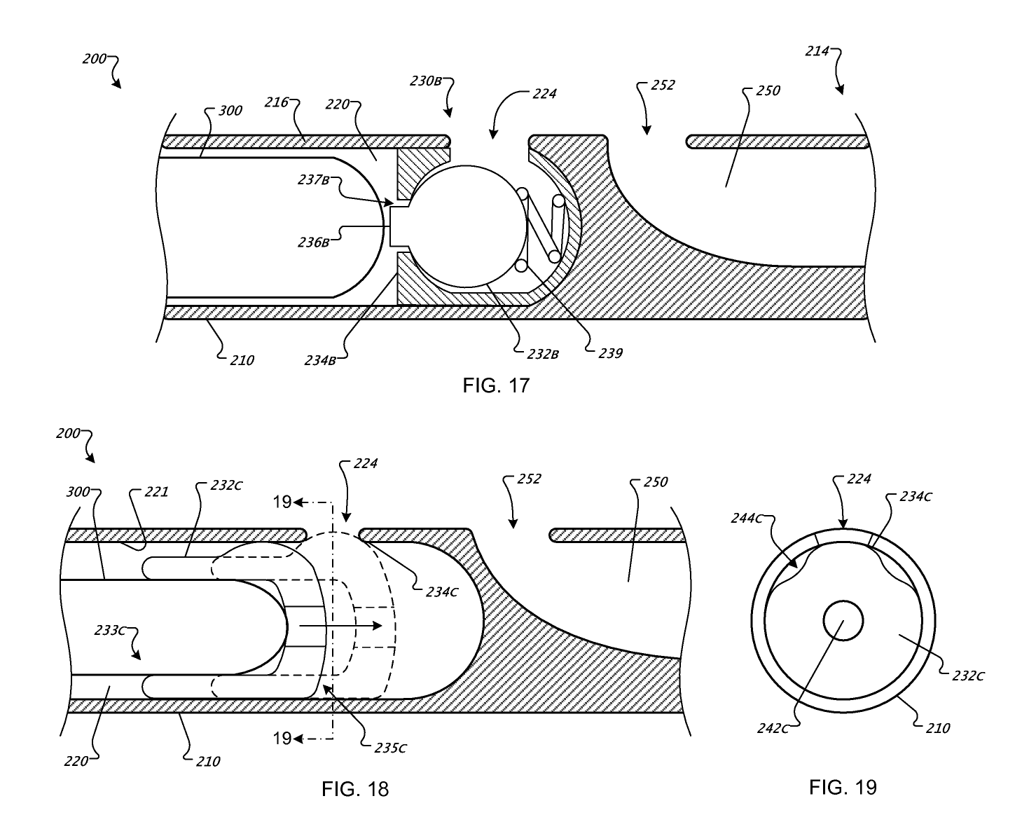

Referring to FIG. 17, in some implementations, the catheter 200 includes a valve 230B having a valve seat 234B comprising an orifice defined by the catheter body 210 in the working lumen 220. A spring 239 biases the valve element 232B, which is disposed distally of the orifice 237B, into sealing engagement with the valve seat 234B to occlude the orifice 237B. In some examples, a valve element operator 236B extends proximally through the orifice 237B to enable actuation of the valve 230B by the received deliverable instrument 300. In the example shown, the catheter body 210 defines a guide wire port 252 adjacent to the valve 230B and in fluid communication with a guide wire lumen 250. The working lumen 220 terminates at the port 224 defined, in this case, in the catheter body sidewall 216, and is not in fluid communication with the guide wire lumen 250.

In the example illustrated in FIGS. 18-19, the catheter 200 includes a valve element configured as a lumen plug 232C that slides axially through the working lumen 220. The lumen plug 232C is configured to be moved between a first position, in which it permits fluid flow through the second port 224, and a second position, in which it forms a seal with a valve seat 234C defined by a wall 221of the working lumen 220, occluding the second port 224. In some instances, the lumen plug 232C defines a fluid channel 242C (see FIG. 19) extending from a longitudinal proximal end 233C of the lumen plug 232C to a distal end 235C of the lumen plug 232C. In other examples, the lumen plug 232C defines at least one fluid channel 244C along an outer surface 246C of the lumen plug 232C. The fluid channel(s) 242C, 244C is in fluid communication with the second port 224 when the lumen plug 232C is in the first position.

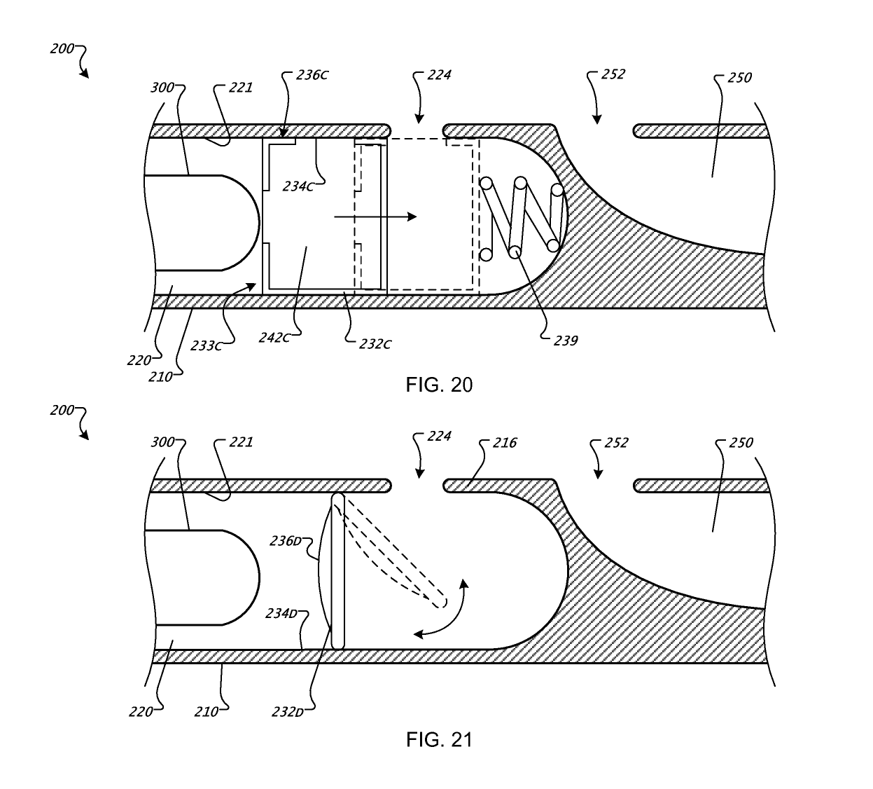

As shown in FIG. 20, the lumen plug 232C may be configured to move between a first position in sealing engagement with a valve seat 234C defined by a wall 221 of the working lumen 220, thereby occluding the working lumen 220, and a second position that permits fluid flow through the second port 224. In this example, the defined fluid channel 242C extends from a longitudinal proximal end 233C of the lumen plug 232C to a side 236C of the lumen plug 232C. A spring 239 biases the lumen plug 232C toward sealing engagement with the valve seat 234C.

In the examples illustrated in FIGS. 14 and 21, the catheter 200 includes a valve element 232D (e.g. a gate) pivotally attached to the catheter body 210 in the working lumen 220. The gate type valve element 232D swings between a first position (or state), in sealing engagement with the valve seat 244D, and a second position (or state) in response to movement of the received deliverable instrument 300. In some examples, the working lumen 220 terminates at the port 224 defined, in this case, in the catheter body sidewall 216. As a result, the port 224 is not in fluid communication with the guide wire lumen 250. The valve element 232D may extend along the wall 221 of the working lumen 220 for sealing engagement with the valve seat 244D defined by the lumen wall 221. A valve element operator 236D extends at least partially across the working lumen 220 for actuation by the received deliverable instrument 300.

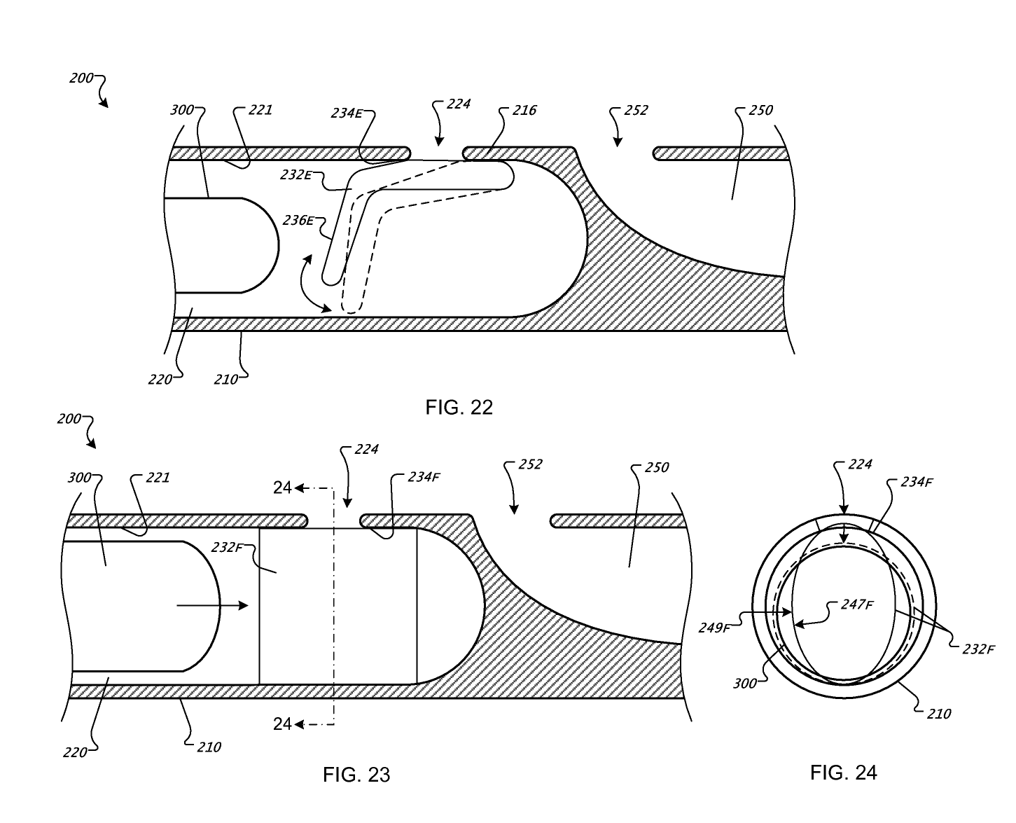

In another example, illustrated in FIG. 22, the catheter 200 includes an elastically deformable valve element 232E disposed in the working lumen 220. The valve element 232E defines a valve element operator 236E, such as an arm, that the received deliverable instrument 300 engages to elastically deform the valve element 232E. When moved distally, the received deliverable instrument 300 elastically deforms the valve element 232E, thus causing it to transition from a first state or position, in sealing engagement with a valve seat 234E defined by the lumen wall 221, to a second state or position, in which it permits fluid flow through the second port 224.

Referring to the example illustrated in FIGS. 23-24, the catheter 200 includes a tubular valve element 232F having an ovular cross-section. The tubular valve element 232F has an inner wall 247F and an outer wall 249F. The received deliverable instrument 300 engages the inner wall 247F of the tube 232F to elastically alter a geometry of the tube 232F from a first configuration, in which the tubular valve element 232F is in sealing engagement with a valve seat 234F defined by the lumen wall 221, to a second configuration, in which the tubular valve element 232F permits fluid flow though the second port 224. In the example shown, the tube 232F deforms from an ovular shape to a circular shape. The tube 232F may be secured to the lumen wall 221 at a location substantially opposite the second port 224, as shown. In other examples, the tube 232F is held in place to engage the valve seat 234F by retaining elements (e.g. shoulders) (not shown) defined by the working lumen 220. In yet more examples, the tube 232F is held in place to engage the valve seat 234F by a tether (not shown) attached to the lumen wall 221. The tubular valve element 232F may have other cross-sectional shapes, such as triangular, square, or some other polygonal shape.

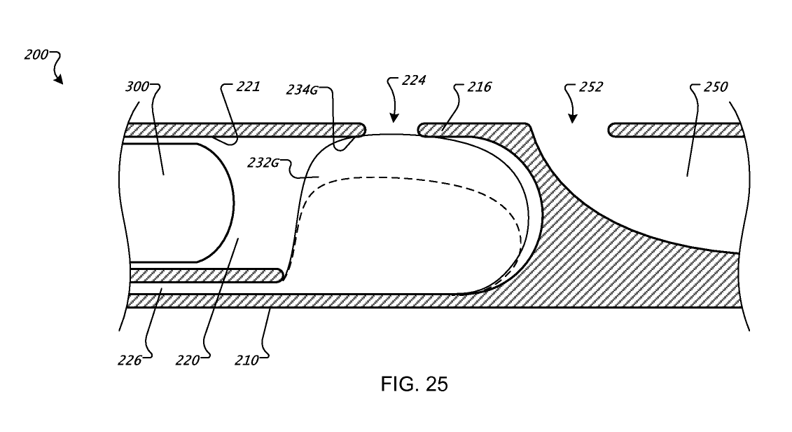

In another example, illustrated in FIG. 25, the catheter 200 includes a valve element 232G configured as an inflatable bladder disposed in the working lumen 220. The bladder 232G transitions between an inflated state, in which it forms a seal with a valve seat 234G defined by the lumen wall 221, and a deflated state, in which it allows fluid flow through the second port 224. In the example shown, the catheter body 110 defines a bladder inflation channel 226 in fluid communication with the bladder 232G to control bladder inflation. The working lumen 220 may terminate at the port 224 defined, in this case, in the catheter body sidewall 216. In such cases, the working lumen 220 is not in fluid communication with the guide wire lumen 250.

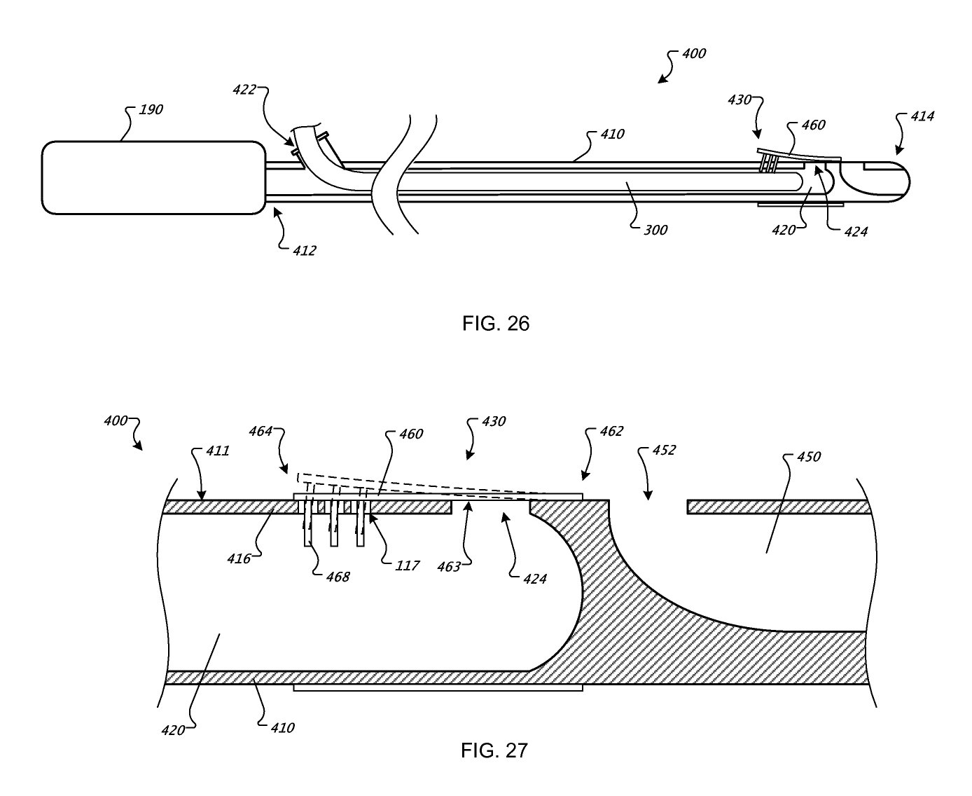

Referring to FIGS. 26-27, a catheter 400 includes a longitudinally extending body 410 having proximal and distal ends, 412 and 414 respectively, and defining at least one lumen 420. A handle 190 may be disposed at the proximal end 412 for holding and manipulating the catheter 400. The lumen 420 extends longitudinally from a first port 422, defined at the proximal end 412, through the body 410 to a second port 424 defined at the distal end 414. A valve 430 in fluid communication with the second port 424 controls fluid flow through the second port 424. The valve 430 includes an elastic tube 460 surrounding the catheter body 410 in a first configuration in sealing engagement with an outer surface 411 of the catheter body 410 to inhibit fluid flow through the second port 424. The valve 430 includes at least one projection 468 extending radially inward from an inner surface 463 of the tube 460. Each projection 468 extends into the lumen 420 through an aperture 117 defined by the catheter body 410. In some examples, the aperture 117 comprises the second port 424. Each projection 468 is configured such that the deliverable instrument 300 received by the lumen 420 displaces the projection 468 out of the lumen 420, thereby distending the tube 460 away from the outer surface 411 of the catheter body 410 and causing the tube 460 to assume a second configuration, in which it permits fluid flow through the second port 424.

The elastic tube 460 has a first end 162 distal to the second end 164 on the catheter body 410 and bonded, heat sealed, ultrasonically welded, or otherwise attached to the catheter body 410. In the example shown, the catheter body 410 defines a guide wire port 452 adjacent to the valve 430 and in fluid communication with a guide wire lumen 450. The lumen 420 terminates at the port 424 defined, in this case, in the catheter body sidewall 416. As a result, the lumen 420 is not in fluid communication with the guide wire lumen 450. In some implementations, a non-elastic tube 460 transitions between open and closed states over the port 424.

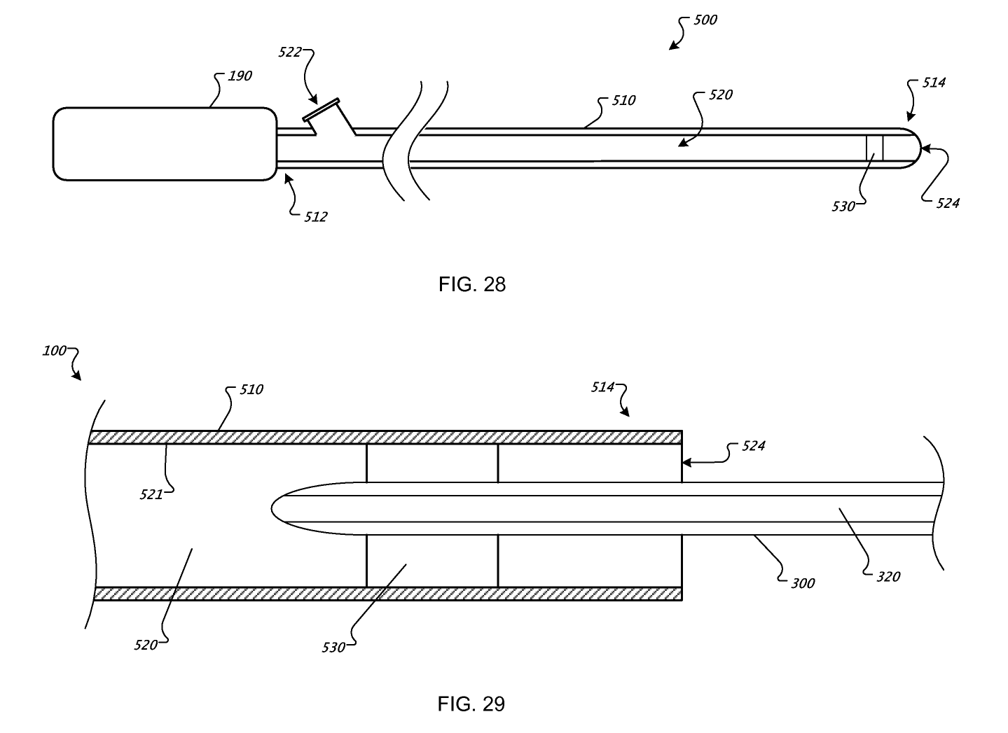

Referring to FIGS. 28-29, in some implementations, a catheter 500 includes a longitudinally extending body 510 having proximal and distal ends 512, 514 and defines at least one lumen 520. A handle 190 may be disposed at the proximal end 512 for holding and manipulating the catheter 500. The lumen 520 extends longitudinally from a first port 522, defined at the proximal end 512, through the body 510 to a second port 524 defined at the distal end 514. A self-sealing fluid barrier 530 extends across the lumen 520 in fluid communication with the second port 524 to control fluid flow through the second port 524. The self-sealing fluid barrier 530 is configured to receive a deliverable instrument 300 pierced therethough. The received deliverable instrument 300, in this example, defines an instrument lumen 320 that allows fluid flow therethrough. Exemplary instruments include syringes and stylettes.

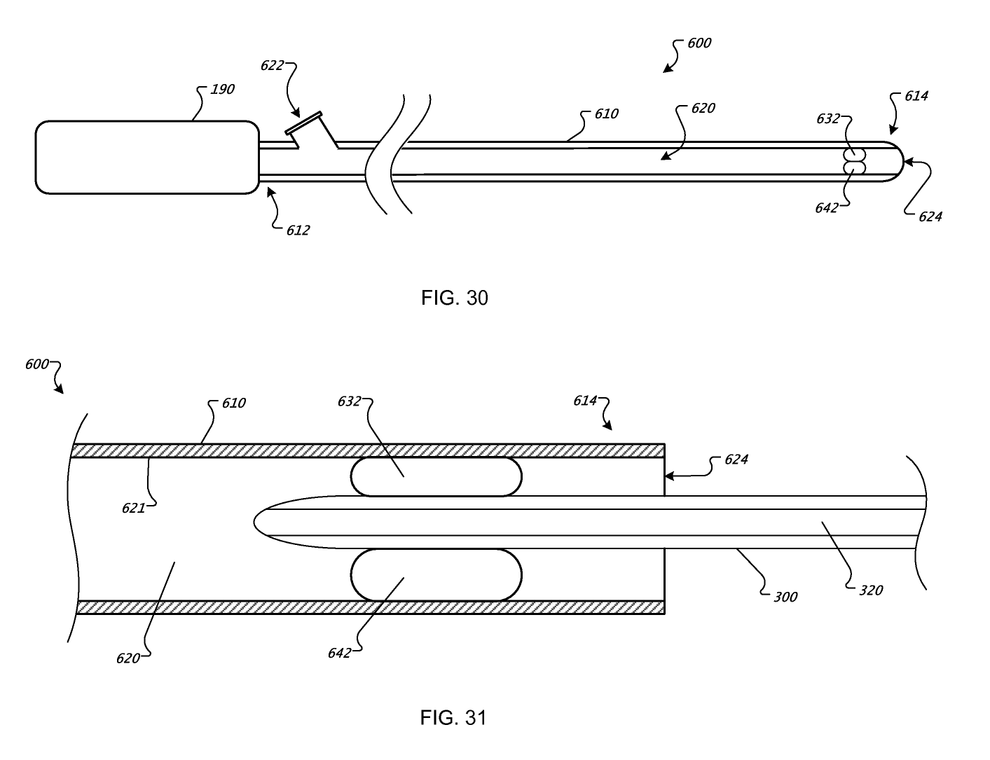

Referring to FIGS. 30-31, in some implementations, a catheter 600 includes a longitudinally extending body 610 having proximal and distal ends 612, 614 and defines at least one working lumen 620. A handle 190 may be disposed at the proximal end 612 for holding and manipulating the catheter 600. The working lumen 620 extends longitudinally from a first port 622, defined at the proximal end 612, through the body 610 to a second port 624 defined at the distal end 614. A valve 630 in fluid communication with the second port 624 controls fluid flow therethrough. The valve 630 includes first and second compliant bladders 632 and 642, respectively, mounted opposite each other in the working lumen 620. The compliant bladders 632, 642 together form a seal to inhibit fluid flow through the second port 624. The bladders 632, 642 are configured to receive a deliverable instrument 300 therebetween. The received deliverable instrument 300, in this example, defines an instrument lumen 320 that allows fluid flow therethrough.

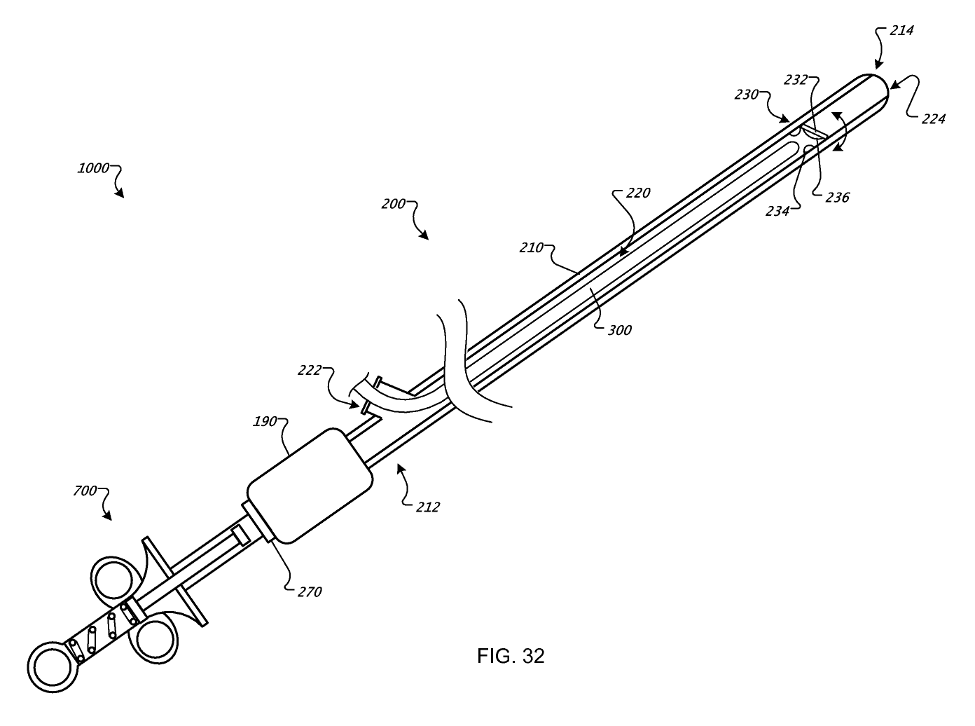

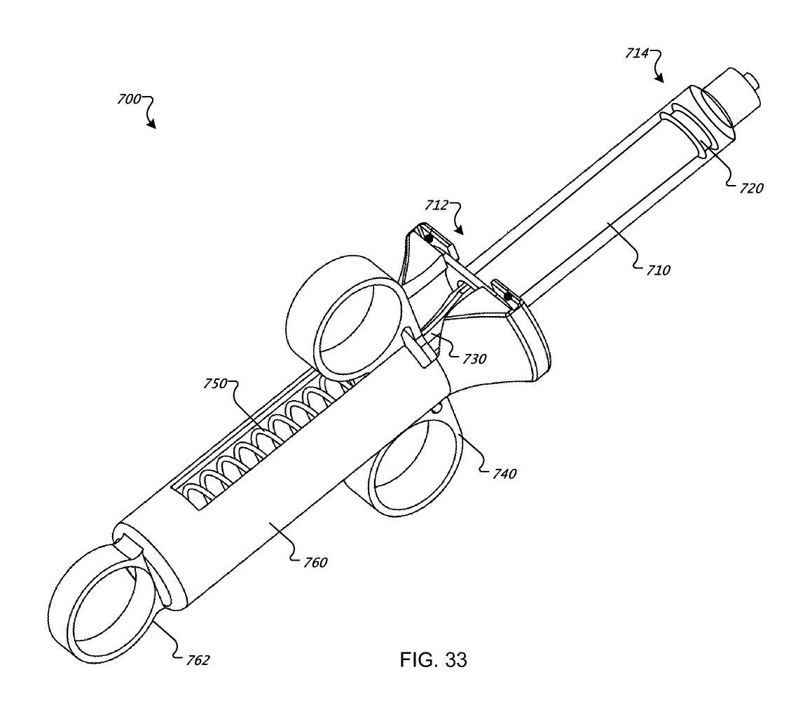

Referring now to FIGS. 32-33, in some implementations, a catheter system 1000 includes a catheter 200 and a catheter primer 700 to flush the catheter 100 with saline prior to and/or during use. A primer connector 270 (e.g. a Luer fitting) disposed at the proximal end 212 of the catheter 200 (e.g. on the handle or hub 190) and in fluid communication with the working lumen 220 receives the catheter primer 700 for delivery of saline into the working lumen 220. The saline is flushed through the working lumen 220 and out the second port 224.

The catheter primer 700, shown in FIG. 33, includes a barrel 710 having an open proximal end 712 and an open distal end 714. The barrel 710 may be a rotationally symmetrical barrel in the shape of a hollow cylinder. A plunger 720 is configured to be receivable in the barrel 710 and movable therealong. A shank 730 attached to the plunger actuates the plunger 720 longitudinally inside along barrel 710. A handle 740 is secured to the barrel 710 and is configured to be grasped by a user. A spring 750 biases the shank 730 and associated plunger 720 into the proximal end 712 of the barrel 710 toward the distal end 714 of the barrel 710. In the example shown, a spring retainer 760 is coupled to the proximal end 712 of the barrel 710 and is configured to retain the spring 750. The spring retainer 760 may define a portion 762 conducive for being grasped (e.g. a finger loop, as shown). The spring biased shank 730 and associated plunger 720 may provide a constant (or non-constant) pressure on fluid held in the barrel 710. For example, saline held in the barrel 710 is pressurized to flow out of the distal end of the barrel into the working lumen 220, thereby flushing the catheter 200.

A number of implementations have been described. Nevertheless, it will be understood that various modifications may be made without departing from the spirit and scope of the disclosure. Accordingly, other implementations are within the scope of the following claims.

What is claimed is:

1. A catheter comprising:
a longitudinally extending catheter body having proximal and distal ends, the catheter body defining a lumen extending longitudinally from a first port defined at the proximal end, through the body, to a second port defined at the distal end; and
a valve for controlling fluid flow through the second port.

2. The catheter of claim 1, wherein the valve comprises:
a valve element pivotally attached to the catheter body and extending into the lumen; and
a valve seat defined by the catheter body in the lumen and configured to receive the valve element;
wherein the valve element is configured to transition between a first state in sealing engagement with the valve seat, and a second state spaced from the valve seat for permitting fluid flow through the second port in response to pressure in the lumen.

3. The catheter of claim 2, wherein the valve element is biased to maintain the first state.

4. The catheter of claim 1, wherein the valve comprises:
a compliant flap secured to the catheter body in the lumen; and
a valve seat defined by the catheter body in the lumen and configured to receive the flap;
wherein the flap is configured to maintain a first state in sealing engagement with the valve seat and to elastically deflect into a second state spaced from the valve seat for permitting fluid flow through the second port when pressure in the lumen exceeds a threshold pressure.

5. The catheter of claim 1, wherein the valve comprises at least first and second compliant flaps secured opposite each other in the lumen, the flaps being elastically deformable to move between a first state in sealing engagement with each other, inhibiting fluid flow through the second port, and a second state allowing fluid flow through the second port out of the lumen when pressure in the lumen exceeds a threshold pressure.

6. The catheter of claim 5, wherein the first and second compliant flaps each have proximal and distal ends with a broad side extending therebetween, the proximal ends of the flaps secured circumferentially to the inner wall of the lumen, the flaps extending toward each other and meeting along the broad sides when in the first position.

7. The catheter of claim 1, wherein the valve comprises an elastic tube surrounding the catheter body in a first state in sealing engagement with an outer surface of the catheter body to temporarily seal the second port, the tube being configured to distend into a second state that allows fluid flow out of the second port when pressure in the lumen exceeds a threshold pressure.

8. The catheter of claim 7, wherein the elastic tube has proximal and distal ends, the distal end being secured to the catheter body.

9. The catheter of claim 1, wherein the valve comprises
a valve element disposed in the lumen for movement;
a valve seat defined by the catheter body in the lumen; and
a spring for biasing the valve element toward a first position in sealing engagement with the valve seat;
wherein the valve element is operable to move from the first position in sealing engagement with the valve seat to a second position spaced from the valve seat for permitting fluid flow through the second port.

10. The catheter of claim 9, wherein the valve element moves from the first position to the second position when pressure in the lumen exceeds a threshold pressure.

11. The catheter of claim 1, wherein the valve comprises first and second compliant bladders mounted opposite each other in the lumen and disposed in a first position in sealing engagement with each other to inhibit fluid flow through the second port, wherein the first and second compliant bladders move to a second position for permitting fluid flow between the bladders and through the second port when pressure in the lumen exceeds a threshold pressure.

12. The catheter of claim 1, wherein the valve comprises a membrane disposed across the lumen, the membrane defining an aperture configured to remain closed in a first position to inhibit fluid flow through the second port, the aperture opening to a second position for permitting fluid flow through the second port when pressure in the lumen exceeds a threshold pressure.

13. The catheter of claim 1, wherein the catheter body defines at least one axial groove in a wall of the lumen, the groove extending from the proximal end of the catheter body to the distal end of the catheter body.

14. The catheter of claim 1, wherein the catheter body defines at least one axial protrusion in a wall of the lumen, the protrusion extending from the proximal end of the catheter body to the distal end of the catheter body.

15. A catheter comprising:
a longitudinally extending catheter body having proximal and distal ends, the catheter body defining a first lumen extending longitudinally between a first port, defined at the proximal end, and a second port, defined at the distal end, and a second lumen extending from a side of the catheter body to the distal end of the catheter body, the second port providing fluid communication between the first and second lumens; and
an elastomeric tube disposed in the second lumen in a first state in sealing engagement with a wall of the second lumen to inhibit fluid flow through the second port;
wherein the tube elastically deflects to a second state spaced from the wall of the second lumen for permitting fluid flow through the second port when pressure in the first lumen exceeds a threshold pressure.

16. A catheter comprising:
a longitudinally extending catheter body having proximal and distal ends, the catheter body defining a lumen extending longitudinally from a first port, defined at the proximal end, through the body to a second port, defined at the distal end; and
a filter disposed in the lumen for controlling fluid flow through the second port.

17. The catheter of claim 16, wherein the filter defines fluid passageways therethrough having a diameter less than about 8 microns.

18. The catheter of claim 16, wherein the filter is configured to prevent blood flow therethrough.

19. A catheter comprising:
a longitudinally extending catheter body having proximal and distal ends, the catheter body defining a lumen extending longitudinally from a first port, defined at the proximal end, through the body to a second port, defined at the distal end, and a valve seat; and
a valve in fluid communication with the second port controls fluid flow through the second port, the valve having:
a valve element disposed within the lumen for transition between a first state in sealing engagement with the valve seat to inhibit fluid flow through the second port, and a second state spaced from the valve seat for permitting fluid flow through the second port; and
a valve element operator that, when engaged by a deliverable instrument received by the lumen, causes a transition of the valve element from the first position to the second position.

20. The catheter of claim 19, wherein the valve element is secured in the lumen to the catheter body and extends circumferentially along a wall of the lumen over the second port to a distal tip, the valve element being torsionally biased against the lumen wall in sealing engagement with the valve seat defined by the lumen wall, wherein circumvolution of the valve element operator disposed at the distal tip of the valve element causes the valve element to elastically deform from the first position to the second position, permitting fluid flow through the second port.

21. The catheter of claim 19, wherein a distal tip of the received deliverable instrument is configured to engage the valve element operator, such that torsional movement of the received deliverable instrument is translated to the valve element operator.

22. The catheter of claim 19, wherein the valve seat comprises an orifice defined by the catheter body in the lumen, the valve element being disposed distally of the orifice and spring biased toward sealing engagement with the valve seat to occlude the orifice.

23. The catheter of claim 22, wherein the valve element operator extends through the orifice for actuation of the valve by the received deliverable instrument.

24. The catheter of claim 19, wherein the valve element comprises a lumen plug slidably disposed within the lumen for movement between a first position that permits fluid flow through the second port, and a second position in sealing engagement with the valve seat defined by a wall of the lumen, occluding the lumen.

25. The catheter of claim 24, wherein the lumen plug defines a fluid channel extending from a longitudinal proximal end of the lumen plug to a lateral side of the lumen plug, the fluid channel being in fluid communication with the second port when the lumen plug is in the first position.

26. The catheter of claim 24, wherein the lumen plug is biased by a spring toward sealing engagement with the valve seat.

27. The catheter of claim 19, wherein the valve element comprises a gate pivotally attached to the catheter body in the lumen, the gate swinging from the first position, in sealing engagement with the valve seat, to the second position when moved by the received deliverable instrument.

28. The catheter of claim 27, wherein a side wall of the catheter body defines the second port, the valve element extending along a wall of the lumen for sealing engagement with the valve seat defined by the lumen wall, the valve element operator extending at least partially across the lumen for actuation by the received deliverable instrument.

29. The catheter of claim 19, wherein the valve element is disposed in the lumen and elastically deformed by the deliverable instrument when moved from the first position, in sealing engagement with the valve seat, to the second position, permitting fluid flow through the second port.

30. The catheter of claim 19, wherein the valve element comprises an ovular tube having inner and outer walls, a portion of the inner wall of the tube defining the valve element operator, the received deliverable instrument engaging the valve element operator to elastically alter a geometry of the tube to the second position for permitting fluid flow though the second port.

31. The catheter of claim 19, wherein the valve element comprises an inflatable bladder disposed in the lumen, the bladder being operable to distend between a first position in sealing engagement with the valve seat and the second position for allowing fluid flow through the second port.

32. The catheter of claim 31, wherein the catheter body defines a bladder inflation channel in fluid communication with the bladder to control bladder inflation.

33. The catheter of claim 19, wherein the catheter body defines at least one axial groove in a wall of the lumen, the groove extending from the proximal end of the catheter body to the distal end of the catheter body.

34. The catheter of claim 19, wherein the catheter body defines at least one axial protrusion in a wall of the lumen, the protrusion extending from the proximal end of the catheter body to the distal end of the catheter body.

35. A catheter comprising:
a longitudinally extending catheter body having proximal and distal ends, the catheter body defining a lumen extending longitudinally from a first port, defined at the proximal end, through the body to a second port, defined at the distal end; and
a valve for controlling fluid flow through the second port, the valve having:
an elastic tube surrounding the catheter body in a first state for sealing an outer surface of the catheter body to inhibit fluid flow through the second port; and
at least one projection extending radially inwardly from an inner surface of the tube, the projection extending into the lumen through an aperture defined by the catheter body, the projection being configured to be displaced in a radially outward direction by a deliverable instrument received by the lumen, thereby pushing a portion of the tube away from the outer surface of the catheter body to a second position to permit fluid flow through the second port.

36. The catheter of claim 35, wherein the aperture comprises the second port.

37. The catheter of claim 35, wherein the tube has first and second ends, the first end disposed distally of the second end on the catheter body and secured to the catheter body.

38. A catheter comprising:
a longitudinally extending catheter body having proximal and distal ends, the catheter body defining a lumen extending longitudinally from a first port, defined at the proximal end, through the body to a second port, defined at the distal end; and
a self-sealing fluid barrier disposed in the lumen for controlling fluid flow through the second port, the self-sealing fluid barrier configured to be pierced a deliverable instrument.

39. The catheter of claim 38, wherein the self-sealing fluid barrier extends across the lumen.

40. The catheter of claim 38, wherein the received deliverable instrument defines a lumen therethrough that allows fluid flow.

41. A catheter comprising:
a longitudinally extending catheter body having proximal and distal ends, the catheter body defining a lumen extending longitudinally from a first port, defined at the proximal end, through the body to a second port, defined at the distal end; and
a valve for controlling fluid flow through the second port, the valve including first and second compliant bladders mounted opposite each other in the lumen and disposed in a first position in sealing engagement to inhibit fluid flow through the second port, the bladders being configured to receive a deliverable instrument therebetween.

42. The catheter of claim 41, wherein the received deliverable instrument defines a lumen therethrough that allows fluid flow.

43. A catheter comprising:
a longitudinally extending catheter body having proximal and distal ends, the catheter body defining a lumen extending longitudinally from a first port, defined at the proximal end, through the body to a second port, defined at the distal end; and
a means for selectively controlling fluid flow through the second port.

44. A catheter system comprising:
a catheter including:
a longitudinally extending catheter body having proximal and distal ends, the catheter body defining a lumen extending longitudinally from a first port, defined at the proximal end, through the body to a second port, defined at the distal end; and
a valve for controlling fluid flow through the second port, the valve having:
a valve element disposed within the lumen for transition between a first state in sealing engagement with a valve seat defined by the catheter body to inhibit fluid flow through the second port, and a second state in which the valve element is spaced from the valve seat to permit fluid flow through the second port; and
a valve element operator defined by the valve element and configured for engagement by a deliverable instrument received by the lumen to cause a transition of the valve element between the first state to the second state; and
a catheter primer in fluid communication with the catheter lumen, the catheter primer comprising:
a barrel having open proximal and distal ends;
a plunger receivable in the barrel and movable therealong;
a shank attached to the plunger and configured to actuate the plunger;
a handle secured to the barrel; and
a spring for biasing the shank toward the distal end of the barrel.

45. The catheter system of claim 44, wherein the catheter primer further comprises a spring retainer coupled to the proximal end of the barrel and configured to retain the spring.

46. The catheter system of claim 44, wherein the valve element is secured in the lumen to the catheter body and extends circumferentially along a wall of the lumen over the second port to a distal tip, the valve element being torsionally biased against the lumen wall in sealing engagement with the valve seat defined by the lumen wall, wherein circumvolution of the valve element operator disposed at the distal tip of the valve element causes the valve element to elastically deform from the first position to the second position, permitting fluid flow through the second port.

47. The catheter system of claim 44, wherein a distal tip of the received deliverable instrument is configured to engage the valve element operator, such that torsional movement of the received deliverable instrument is translated to the valve element operator.

48. The catheter system of claim 44, wherein the valve seat comprises an orifice defined by the catheter body in the lumen, the valve element being disposed distally of the orifice and spring biased toward sealing engagement with the valve seat to occlude the orifice.

49. The catheter system of claim 44, wherein the valve element operator extends through the orifice for actuation of the valve by the received deliverable instrument.

50. The catheter system of claim 44, wherein the valve element comprises a lumen plug slidably disposed within the lumen for movement between a first position that permits fluid flow through the second port, and a second position in sealing engagement with the valve seat defined by a wall of the lumen, occluding the lumen.

51. The catheter system of claim 50, wherein the lumen plug defines a fluid channel extending from a longitudinal proximal end of the lumen plug to a lateral side of the lumen plug, the fluid channel being in fluid communication with the second port when the lumen plug is in the first position.

52. The catheter system of claim 50, wherein the lumen plug is biased by a spring toward sealing engagement with the valve seat.

53. The catheter system of claim 44, wherein the valve element comprises a gate pivotally attached to the catheter body in the lumen, the gate swinging from the first position, in sealing engagement with the valve seat, to the second position when moved by the received deliverable instrument.

54. The catheter system of claim 53, wherein a side wall of the catheter body defines the second port, the valve element extending along a wall of the lumen for sealing engagement with the valve seat defined by the lumen wall, the valve element operator extending at least partially across the lumen for actuation by the received deliverable instrument.

55. The catheter system of claim 44, wherein the valve element is disposed in the lumen and elastically deformed by the deliverable instrument when moved from the first position, in sealing engagement with the valve seat, to the second position, permitting fluid flow through the second port.

56. The catheter system of claim 44, wherein the valve element comprises an ovular tube having inner and outer walls, a portion of the inner wall of the tube defining the valve element operator, the received deliverable instrument engages the valve element operator to elastically alter a geometry of the tube to the second position for permitting fluid flow though the second port.

57. The catheter system of claim 44, wherein the valve element comprises an inflatable bladder disposed in the lumen, the bladder being operable to distend between a first position in sealing engagement with the valve seat and the second position for allowing fluid flow through the second port.

58. The catheter system of claim 57, wherein the catheter body defines a bladder inflation channel in fluid communication with the bladder to control bladder inflation.

59. The catheter system of claim 44, wherein the catheter body defines at least one axial groove in a wall of the lumen, the groove extending from the proximal end of the catheter body to the distal end of the catheter body.

60. The catheter system of claim 44, wherein the catheter body defines at least one axial protrusion in a wall of the lumen, the protrusion extending from the proximal end of the catheter body to the distal end of the catheter body.

61. A catheter comprising:
a longitudinally extending catheter body having proximal and distal ends, the catheter body defining a lumen extending longitudinally from a first port, defined at the proximal end, through the body to a second port, defined at the distal end;
wherein a distal portion of the body defines a valve for controlling fluid flow through the second port, the distal portion of the body being formed in a substantially U-shaped cross-section having first and second substantially U-shaped flaps joined along lateral edges; and
wherein the flaps are configured to move between a first state permitting fluid flow through the second port and a second state in sealing engagement with each other when pressure in the lumen drops below a threshold pressure.

62. The catheter of claim 61, further comprising a guide wire tube disposed over the body and positioned to define a guide wire opening with a transition section of the body in between the valve and a main section of the body.

63. The catheter of claim 61, further comprising a reinforcing member embedded in the body at least along the distal portion of the body.

64. The catheter of claim 63, wherein the reinforcing member comprises a rail disposed substantially along the lateral edges of the flaps.

65. The catheter of claim 63, wherein the reinforcing member comprises a coil.

66. The catheter of claim 63, wherein the reinforcing member comprises braids.
